(19) United States
(12) Patent Application Publication
(10) Pub. No.: US 2009/0175806 A1
(43) Pub. Date: Jul. 9, 2009

(54) Antimicrobial Compositions Containing Low Concentrations of Botanicals

(76) Inventors: Shanta Modak, River Edge, NJ (US); Nayana Baiju, New York, NY (US); Lauserpina Caraos, Hollis, NY (US)

Correspondence Address: BAKER BOTTS L.L.P., 30 ROCKEFELLER PLAZA, 44TH FLOOR, NEW YORK, NY, 10112-4498, US

(21) Appl. No.: 12/367,851

(22) Filed: Feb. 9, 2009

Related U.S. Application Data
(63) Continuation-in-part of application No. PCT/US08/72006, filed on Aug. 1, 2008.
(60) Provisional application No. 60/953,654, filed on Aug. 2, 2007.

(51) Int. Cl. A01N 65/00; A01N 65/36; A01N 65/24; C11D 3/382; A61P 17/10; A61K 36/752; A61K 36/54; A61K 36/87; A61K 36/00; A61Q 9/00; A61Q 11/00; A61Q 17/04

(52) U.S. Cl. 424/58; 424/725; 424/736; 424/739; 424/766; 510/138; 510/141; 424/73; 424/59

(57) ABSTRACT

The present invention relates to a preservative or antimicrobial compositions with broad spectrum antimicrobial activity comprising low concentrations of essential oil (and/or one or more component thereof) and a botanical extract in synergistic combination with a fruit acid and alkanediol, and optionally a solvent. The compositions of the invention may be used in personal care products such as creams or soap products.

### PRIORITY CLAIMED

This application is a continuation in part application of International Patent Application No. PCT/US08/72006, filed Aug. 1, 2008, which claims priority to U.S. patent application Ser. No. 12/134,918, filed Jun. 6, 2008; to U.S. patent application Ser. No. 12/016,788, filed Jan. 18, 2008; which claims priority to U.S. Provisional Application Ser. Nos. 60/953,654, filed Aug. 2, 2007, and 60/945,288, filed Jun. 20, 2007, the disclosures of which are hereby incorporated by reference in their entireties herein.

### 1. INTRODUCTION

The present invention relates to broad spectrum antimicrobial and preservative compositions containing combinations of low concentrations of including one or more essential oil (and/or one or more component thereof), botanical extracts, including plant extracts and fruit extracts, in synergistic combinations with one or more fruit acids and alkanediols. The compositions of the invention may be used as non-toxic alternatives to conventional disinfectants or may be combined with other antimicrobial agents to enhance their activity. The invention provides effective alternatives to harsher products, and may be particularly useful in personal care and household product applications and where children and/or pet exposure may be a concern.

### 2. BACKGROUND OF THE INVENTION

Essential oils are volatile oils obtained from plant or animal sources and are composed of complex mixtures of several constituents, such as monoterpenes and sesquiterpene hydrocarbons, monoterpene and sesquiterpene alcohols, esters, ethers, aldehydes, ketones, oxides and the like. These essential oils and their isolated constituents are frequently utilized as fragrance and flavor agents, and have been widely used in folk medicine for wound healing properties.

Scientific research has corroborated the beneficial effects of essential oils. Essential oils of eucalyptus have been found to “possess central and peripheral analgesic effects as well as neutrophil-dependent and independent anti-inflammatory activities” (Silva et al., 2003, J. Ethnopharmacol. 89(2-3); 277-283), and similar activity has been observed in essential oils from Lavendula angustifolia Mill. (Hajhashemi et al., 2003, J. Ethnopharmacol. 89(1):67-71). Essential oils have been demonstrated to exhibit antibacterial (Bezic et al., 2003, Phytother. Res. 17(9:1037-1040; Goren et al., 2003, Z. Naturforsch. 58(9-10):687-690; de Abreu Gonzaga et al., 2003, Planta Med. 69(8:773-775; Valero and Salmera, 2003, Int. J. Food Microbiol. 85(1-2): 73-81) and antifungal (Paranagama et al., 2003, Lett. Appl. Microbiol. 37(1):86-90; Shin, 2003, Arch. Pharm. Res. 26(5):389-393; Velluti et al., 2003, Int. J. Food Microbiol. 89:145-154) activities. Virucidal activity of essential oils has also been observed, including direct virucidal effects against Herpes simplex viruses types 1 and 2 (Garcia et al., Phytother. Res. 17(9):1073-1075; Minami et al., 2003, Microbial Immunol. 47(a):681-684; Schuhmacher et al., 2003, Phytomedicine 10:504-510).

United States Patent Application Publication No. 20050048139 by Modak et al., published Mar. 3, 2005, relates to topical compositions comprising an emollient solvent and an essential oil, which may further comprise additional additives, among which citric acid, glycolic acid and lactic acid are cited. It does not recognize the synergistic activity between essential oils and fruit acids nor does it disclose the concentrations of fruit acids to be used to provide a synergistic effect.

United States Patent Application Publication No. 20050019431 by Modak et al., published Jan. 27, 2005, relates to compositions comprising a quaternary ammonium compound and an essential oil (or active component thereof).

A number of patent applications relate to compositions comprising an essential oil (or component thereof) where zinc salts are added to inhibit irritation associated with essential oils. Examples of such patent applications include United States Patent Application Publication No. 20040102429 by Modak et al., published May 27, 2004 and United States Patent Application Publication No. 20050238602 by Modak et al., published Oct. 27, 2005.

U.S. Pat. No. 6,858,317 by Aamodt et al., issued Feb. 22, 2005, relates to methods for protecting wood from mold and sapstaining fungi which employ a non-toxic mold inhibitor which may be a plant extract such as an essential oil.

U.S. Pat. No. 5,100,652 by Kross et al., issued Mar. 31, 1992, relates to low concentration chlorous-acid generating oral hygience compositions which may comprise an essential oil as a flavoring agent.

U.S. Pat. No. 5,310,546 by Douglas, issued May 10, 1994, relates to a mouthrinse preparation comprising hydrogen peroxide, zinc chloride, sodium citrate, sodium lauryl sulfate, citric acid and ethanol and optionally an essential oil which is a denaturing agent.

BiON offers several skin care products comprising citric acid, botanicals, and other agents for topical use (San Diego, Calif., US).

Johnson et al. (U.S. Pat. No. 6,319,958 and US20020165130) relates to the use of sesquiterpenoids to promote uptake of exogenous antimicrobial compounds. Similarly, a related article discloses the use of sesquiterpenoids, such as nerolidol, farnesol, bisabolol and apritone, in enhancing bacterial permeability and susceptibility to exogenous antimicrobial compounds, suggesting that sesquiterpenoids have a non-specific and general effect (Brehm-Stecher et al. 2003, Antimicrobial Agents and Chemotherapy, 47(10):3357-3360). In particular, Brehm-Stecher et al. report that nerolidol, farnesol, bisabolol and apritone enhanced the susceptibility of S. aureus to the antibiotics erythromycin, gentamicin, vancomycin, ciproflaxin, clindamycin, and tetracycline.

U.S. Pat. No. 4,867,898 by Spaulding et al., issued Sep. 19, 1989, relates to a liquid hard surface cleaner comprising pine oil and organic, oil-soluble acids at a pH from 0-6.

U.S. Pat. No. 6,753,305 by Raso and Caselli, issued Jun. 22, 2004, relates to a hard surface disinfectant comprising up to 20 percent of cinnamon oil or a component thereof, 0.01-5 percent of an organic acid, and optionally an additional essential oil.

International Patent Application Publication No. WO2007/077573 by Mukhopadhyay, published Jul. 12, 2007, relates to antimicrobial compositions comprising an antimicrobial agent, such as triclosan, and a functionalized hydrocarbon, where the functionalized hydrocarbon can be an essential oil, and/or a solvent.

There is a continuing desire for an antimicrobial composition that is non-irritating, safe, and effective for repeated use in various professional and non-professional settings.

### 3. SUMMARY OF THE INVENTION

The present invention relates to skin or surface antimicrobial and preservative compositions with broad spectrum antimicrobial containing low concentrations of one or more essential oil (and/or one or more component (i.e., an “Individual Constituent” or “IC”) thereof), and botanical extracts including plant and fruit extracts, in synergistic combinations of one or more fruit acids and alkanediols. It is based, at least in part, on the discovery that the low concentrations of specific combinations of these ingredients have an unexpected synergistic effect, namely the combinations can confer superior antimicrobial properties on personal care, veterinary, as well as household products.

In preferred, non-limiting embodiments, the compositions of the invention further comprise an alkanediol, particularly a bifunctional fatty alcohol, enhances antimicrobial activity still more. In various non-limiting embodiments, the compositions may include a solvent which includes alcohol, glycols, or vegetable oils.

In various non-limiting embodiments, the compositions of the present invention may include a total stock solution concentrations of essential oils and botanical extracts in concentrations ranging from about 0.5% to about 30% (w/w), preferably from about 2% to about 20% (w/w). In stock solutions, the compositions of the present invention contain from about 0.3% to about 15% (w/w), preferably from about 0.5% to about 6.0% (w/w) essential oil or individual constituent thereof; from about 0.3% to about 30% (w/w), preferably from about 1% to about 20% (w/w), more preferably from about 1% to about 15% (w/w) botanical extracts; from about 5% to about 20% (w/w), preferably from about 10% to about 20% (w/w) fruit acids; from about 1% to about 80% (w/w), preferably from about 20% to about 80% (w/w), more preferably from about 30% to about 80% (w/w), more preferably from about 30% to about 50% (w/w) alkanediols; and from about 0% to about 90% (w/w), preferably from about 0% to about 80% (w/w) solvents. In specific non-limiting embodiments, in final products (such as creams or soaps), the synergistic compositions of the present invention contain low total concentrations of essential oils ranging from about 0.01% to about 0.5% (w/w), more preferably from about 0.02% to about 0.1% (w/w); and botanical extracts are present in final products in amounts ranging from about 0.1 to about 0.5% (w/w); and with fruit acids in amounts ranging from 5% to about 20% (w/w). Solvent systems include alcohol, glycerol, diglycerol, propylene glycol, dipropylene glycol, and vegetable oils.

In various non-limiting embodiments, the present invention may be utilized in personal care products such as soaps, scrubs, cosmetics, topical creams and lotions, wound care products, disinfecting wipes, and veterinary products such as pet shampoos, and therapeutic ointments. The compositions of the invention may be used in concentrations from about 1% to about 5% in personal care products or topical creams. Alternatively, 10% to about 20% of the stock solutions of the compositions may be used in soap formulations. Additionally, the compositions contain no or little to very mild added fragrance.

The compositions of the invention may be used as non-toxic alternatives to conventional disinfectants or may be combined with to other antimicrobial agents to enhance their activity, particularly providing persistent antimicrobial protection without causing skin sensitivity. The invention provides effective alternatives to harsher products which may be particularly useful in personal care and household products and where children and/or pet exposure may be a concern.

### 4. DETAILED DESCRIPTION OF THE INVENTION

For clarity of description, and not by way of limitation, the detailed description of the invention is divided into the following subsections:

(4.1) essential oils;

(4.2) botanical extracts;

(4.3) fruit acids;

(4.4) alkanediols;

(4.5) solvents;

(4.6) combinations of essential oils/ICs and fruit acids;

(4.7) compositions comprising alkanediols;

(4.8) compositions comprising essential oils/ICs, botanical extracts, fruit acids and alkanediols;

(4.9) personal care products;

(4.10) veterinary products;

(4.11) household/industrial products; and

(4.12) preservative compositions.

### 4.1 Essential Oils

Essential oils (“EOs”), as defined herein, are volatile oils obtained from plant or animal sources, or their synthetic equivalents, and are composed of complex mixtures of several constituents as monoterpenes and sesquiterpene hydrocarbons, monoterpene and sesquiterpene alcohols, esters, ethers, aldehydes, ketones, oxides and the like. Examples of EOs include, but are not limited to, cinnamon oil, basil oil, bergamot oil, clary sage oil, ylang-ylang oil, neroli oil, sandalwood oil, frankincense oil, ginger oil, peppermint oil, lavender oil, jasmine absolute, geranium oil bourbon, spearmint oil, clove oil, patchouli oil, rosemary oil, rosewood oil, sandalwood oil, tea tree oil, vanilla oil, lemongrass oil, cedarwood oil, balsam oils, tangerine oil, Hinoki oil, Hiba oil, ginko oil, eucalyptus oil, lemon oil, orange oil, sweet orange oil, pomegranate oil, manuka oil, and calendula oil. In preferred non-limiting embodiments of the invention, the EO is selected from one or more EO from the group consisting of cinnamon oil (bark or leaf), lemongrass oil, citronella oil, basil oil, and orange oil.

Individual constituents (“ICs”) of essential oils may be isolated from the oil (natural) or entirely or partially synthetic, and include, but are not limited to, curcumin, 1-citronellol, α-amylcinnamaldehyde, lyral, geraniol, farnesol, hydroxycitronellal, isoeugenol, eugenol, camphor, eucalyptol, linalool, citral, thymol, limonene and menthol. Further examples of ICs include sesquiterpenoid compounds, which may be the active compounds in the essential oils. Sesquiterpenoid compounds, containing 15 carbons, are formed biosynthetically from three 5-carbon isoprene units. Sesquiterpenoid compounds include, but are not limited to, farnesol, nerolidol, bisabolol, apritone, chamazulene, santalol, zingiberol, carotol, and caryophyllen.

Mixtures of one or more EO, one or more IC, and one or more EO as well as one or more IC, are encompassed by the present invention. In specific non-limiting embodiments of the invention, an IC is selected from the (non-limiting) group consisting of camphor, curcumin, alpha-pinene, constituents of cinnamon leaf oil such as, cinnamaldehyde, cinnamylacetic ester, cinnamic acid, ethyl cinnamate, methyl chavicol, linalool, beta-caryophyllene, and eugenol; constituents of lemongrass oil such as d-limonene, geranyl acetate, nerol, geraniol, citral, and/or myrcene; constituents of citronella oil such as geraniol, citronellol, citronellal, geranyl acetate, limonene, methyl isoueugenol, and/or elemol; components of basil oil such as camphor, limonene, and/or β-selinene; and constituents of orange oil such as α-pinene, sabinene, myrcene, limonene, linalool, citronellal, neral and/or geranial. An EO or IC for use in the invention may be obtained from its natural source or may be chemically synthesized.

In various non-limiting embodiments, low concentrations of essential oils and ICs are used. Specifically, the concentrations of each essential oil or IC in the final products may range from about 0.01% to about 0.5% (w/w), preferably from about 0.02% to about 0.1% (w/w). Essential oils or ICs are present in stock solutions in amounts ranging from about 0.3% to about 15% (w/w), preferably from about 0.5% to about 6.0% (w/w). The total concentrations of essential oils and botanical extracts in stock solutions may range from about 0.5% to about 30% (w/w), preferably from about 2% to about 20% (w/w). These concentrations (and others recited throughout) may be increased in stock solutions intended for dilution, where the above ranges provide for the concentration after dilution.

### 4.2 Botanical Extracts

Botanical extracts, as defined herein, include plant, herbal, and fruit extracts, which are not “essential oils” as noted above. The botanicals utilized herein include but are not limited to Camellia sinensis (green tea), grapes, pomegranate, Echinacea, Centella Asiatica, Elderflower, Irish moss, Mallow, soap bark, Yucca, Clary sage, and mixtures thereof. The botanical utilized to obtain the botanical extract may be obtained from any of the plant parts including the leaves, pulp, seeds, or stems as well as the whole plant. Herbal extracts can be, for example, standardized extracts that are dispersible and/or soluble in aqueous medium.

Examples of herbal extracts include, without limitation, extracts of chamomile, rosemary, aloe, nettle, Centella asiatica, ginkgo biloba, betula, and witch hazel. Such extracts may be delivered in a carrier such as water, propylene glycol, hydroalcohol, glycerine, or butylene glycol. Additional extracts with nutritional quality can be used, including, without limitation, green tea, grape skin, grape seed, grapefruit, grapefruit seed, bilberry, blueberry, Ginkgo biloba, soy isoflavones, black cohosh, St. John's wort, echinacea, chamomile, rosemary, aloe, nettle, and Centella asiatica. Botanical extracts can be obtained from, for example, Active Organics (Lewisville, Tex.), New Age Botanicals (Garland, Tex.), Triarco Industries (Wayne, N.J.), and Aloecorp (Broomfield, Colo.).

Additional Examples of botanical extracts include natural blends of fatty acids which mimic those found in the stratum corneum, mixture of fatty acids with pigments such as carotenes, carotenoids or phytosterols that are known to facilitate repair to damaged skin, and the like. Specific examples of useful botanical extracts include avocado, which contains the sterol sitosterol; carrot, which contains beta carotene; sesame oil which contains a mixture of saturated and unsaturated fatty acids, and brazil nut oil. Because of its broad distribution of fatty acids, extracts such as brazil nut oil, can outperform single fatty acids with respect to incorporation into the lipid lamellar structures. Brazil nut oil (BNO) originates from the harvested fruit from the South American rain forest tree: Bertholletia excelsa.

In various non-limiting embodiments, low concentrations of botanical extracts are used. Specifically, the concentrations in final products may range from about 0.1% to about 0.5%. The total concentrations of essential oils and botanical extracts may range from about 2% to about 20%. Botanical extracts are present in stock solutions in concentrations ranging from about 0.3% to about 30%, preferably from about 1% to about 20% (w/w), more preferably from about 1% to about 15% (w/w).

### 4.3 Fruit Acids

Fruit acids which may be used according to the invention include but are not limited to citric acid, glycolic acid, lactic acid, malic acid, tartaric acid and acetic acid. In preferred non-limiting embodiments of the invention, the fruit acid is citric acid. In other preferred non-limiting embodiments of the invention, the fruit acid is malic acid. In other preferred non-limiting embodiments, the fruit acid is Multifruit BSC from Arch Chemicals. Multifruit BSC is a mixture of lactic, citric, tartaric, glycolic, and malic acid extracted from plants.

A fruit acid for use in the invention may be obtained from its natural source or may be chemically synthesized.

In non-limiting embodiments of the invention, the stock solution concentrations of the fruit acids ranges from about 5% to about 20%, more preferably from about 10% to about 20%.

### 4.4 Alkanediols

In non-limiting embodiments, bifunctional alcohols which may be used according to the present invention are alkanediols. Suitable alkanediols include, but are not limited to, dodecanediol, decanediol, nonanediol, octanediol, heptanediol, hexanediol and pentanediol.

In particular non-limiting embodiments, the alkanediols have a carbon backbone of between 9 and 25 carbon atoms, including but not limited to 1,9 Nonanediol, 1,2-Decanediol, 1,10-Decanediol, 1,11-Undecanediol, 1,2-Dodecanediol, 1,12 Dodecanediol, Cyclododecanediol, 1,13-Tridecanediol, 1,2-Tetradecanediol,1,14-Tetradecanediol, 1,15-Pentadecanediol, 1,16-Hexadecanediol, 1,17-Heptadecanediol, 1,18-Octadecanediol, 1,19-Nonadecanediol, 1,20-Eicosanediol, 1,21-Heneicosanediol, 1,22-Docosanediol, 1,23-Tricosanediol, 1,24-Tetracosanediol, 1,25-Pentacosanediol. The preferred alkanediols are 1,2-Decanediol, 1,10-Decanediol, 1,2-Dodecanediol, 1,12-Dodecanediol, Cyclododecanediol, 1,13-Tridecanediol, 1,2-Tetradecanediol, 1,14-Tetradecanediol and the most preferred alkanediols are 1,2-Decanediol, 1,2-Dodecanediol and 1,2-Tetradecanediol.

In non-limiting embodiments of the invention, the stock solution concentrations of the alkanediols ranges from about 1% to about 80% (w/w), preferably from about 20% to about 80% (w/w), more preferably from about 30% to about 80% (w/w), more preferably from about 30% to about 50% (w/w).

### 4.5 Solvents

In various non-limiting embodiments, the compositions of the present invention may include a solvent including but not limited to water, alcohols, glycols, glycerol, glycerine, diglycerol, propylene glycol, dipropylene glycol, and vegetable oils.

Preferred but non-limiting examples of non-alkanediol alcohols for solubilisation are aliphatic alcohols having carbon atoms about 1 to 8 such as methanol, ethanol, n-propanol, isopropyl alcohol, 2-methyl-2 propanol, hexanol, or combinations thereof. Aromatic alcohols, for example, but not by way of limitation, phenoxyethanol, benzyl alcohol, 1-phenoxy-2propanol, and/or phenethyl alcohol, may also optionally be used in combination with aliphatic alcohols.

The solvents are used in stock solution concentrations ranging from about 0% to about 90% (w/w), preferably from about 0% to about 80% (w/w). Alcohol concentrations range from about 0% to about 90%. Phenoxyethanol concentrations range from about 0% to about 40%. Propylene glycol concentrations range from about 0% to about 80%. Vegetable oil concentrations may range from about 0% to about 50%.

### 4.6 Combinations of Essential Oils/ICs and Fruit Acids

In various non-limiting embodiments, the present invention provides for compositions comprising a combination of one or more essential oil (and/or one or more IC thereof) and one or more fruit acid. Preferably, this combination produces a synergistic anti-microbial effect against at least one microbe selected from the group consisting of Escherichia coli, Pseudomonas aeruginosa, Staphylococcus aureus, methicillin-resistant S. aureus, and Candida albicans (“synergistic” means that the antimicrobial effect of the combination is greater than the sum of the antimicrobial effects of the individual components).

In particular, non-limiting embodiments of the invention, the compositions comprise between about 0.1 and 1.2 percent (weight/weight) or between 0.1 and 1.0 percent (weight/weight) (“w/w”) of one or more essential oils, one or more ICs, or a combination thereof (where a combination is used, the total of essential oil(s) and/or IC(s) is between about 0.1 and 1.0 percent (weight/weight) and between about 0.125 and 2.0 percent (weight/weight) of one or more fruit acid (where more than one fruit acid is used, the total amount of fruit acids present is between about 0.125 and 2.0 percent (weight/weight)). “About” as used in this document means plus or minus 20 percent of the recited value, so that, for example, “between about 0.125 and 1.0 percent” means a range between 0.125÷0.025 and 1.0±0.2.

In particular, non-limiting embodiments, the present invention provides for concentrates of essential oil/IC/fruit acid combinations which are concentrated and may be diluted to provide a composition for personal, household, or industrial use. In such concentrates, the ratio of fruit acid to essential oil(s)/IC(s) (weight/weight) is between about 1 and 16, for example, but not by way of limitation, fruit acid(s): EO(s)/IC(s) of between about 1:1 to 10:1, inclusive (weight/weight).

The present invention further provides for methods of providing an antimicrobial effect to a surface comprising applying, to the surface, an effective amount of a composition as described herein. An antimicrobial effect means killing and/or inhibiting the growth/proliferation of a microbe. In particular non-limiting embodiments of the invention, the microbe is selected from the group consisting of from the group consisting of Escherichia coli, Pseudomonas aeruginosa, Staphylococcus aureus, methicillin-resistant S. aureus, and Candida albicans. In specific non-limiting embodiments, the composition is exposed to the surface for at least 20 seconds, at least 30 seconds, or at least 60 seconds, or at least 5 minutes or at least 10 minutes. In various non-limiting embodiments, the surface may be the a skin or mucosal surface, a household surface (e.g., a surface of a countertop, Table sink, toilet, wall, floor, appliance, window, shower surface, rug, upholstery, fabric, etc.) or an industrial surface (e.g., a surface of a countertop, Table sink, toilet, wall, floor, appliance, window, shower surface, rug, upholstery, fabric, etc.).

In a first set of specific, non-limiting embodiments, the present invention provides for a composition comprising a component selected from the group consisting of cinnamon oil, cinnamaldehyde, eugenol, cinnamylacetic ester, and cinnamic acid, at a concentration of between about 0.1 and 1.2 percent (weight/weight) or between about 0.2 and 0.6 percent (weight/weight), as well as citric acid at a concentration of between about 0.5 and 1.5 percent (weight/weight), optionally further comprising triclosan at a concentration of between about 0.05 and 3 percent (weight/weight) or between about 0.05 and 0.1 percent (weight/weight) (this range, and all ranges herein, inclusive). In certain embodiments, the EO/IC is not cinnamon oil or pine oil or an IC thereof.

In a second set of non-limiting embodiments, the present invention provides for compositions comprising a EO/IC mixture comprising two or more EO or IC from the group consisting of cinnamon oil or an IC thereof, lemongrass oil and/or an IC thereof, orange oil and/or an IC thereof, basil oil and/or an IC thereof, and citronella oil and/or an IC thereof, at a total EO/IC concentration of between about 0.1 and 1 percent (weight/weight); together with one or more fruit acid (preferably citric acid), at a total fruit acid concentration of between about 0.125 and 2 percent (weight/weight); and an alcohol (preferably ethanol at a concentration of between about 5-20 percent (weight/weight), optionally further comprising triclosan at a concentration of between about 0.05 and 3 percent (weight/weight) or between about 0.05 and 0.1 percent (weight/weight), where the ratio of EO/IC to fruit acid is between about 1:1 to about 1:10. In certain embodiments, the EO/IC is not cinnamon oil or pine oil or an IC thereof.

In a third set of non-limiting embodiments, the present invention provides for compositions comprising a EO/IC mixture comprising lemongrass oil and/or an IC thereof, orange oil and/or an IC thereof, and optionally one or more additional EO and/or IC, at a total EO/IC concentration of between about 0.1 and 1 percent (weight/weight); together with one or more fruit acid (preferably citric acid), at a total fruit acid concentration of between about 0.125 and 2 percent; and an alcohol (preferably ethanol) at a concentration of between about 5-20 percent (weight/weight), optionally further comprising triclosan at a concentration of between about 0.05 and 1 percent (weight/weight) or between about 0.05 and 0.3 percent (weight/weight), where the ratio of EO/IC to fruit acid is between about 1:1 to about 1:10.

### 4.7 Compositions Comprising Alkanediols

In non-limiting embodiments, the present invention provides for compositions comprising an essential oil, a fruit acid, an alcohol which is not an alkanediol, and an alkanediol. In particular, non-limiting embodiments, the carbon backbone of the alkanediol has between 9 and 25 carbon atoms.

In particular non-limiting embodiments, the present invention provides for compositions comprising (i) between about 0.2 and 0.7 percent (weight/weight) of one or more essential oil as set forth above and preferably selected from the group consisting of lemongrass, cinnamon oil, citronella oil, basil oil, orange oil and combinations thereof; (ii) a non-alkanediol alcohol solvent at a concentration between about 0.5 and 20 percent (weight/weight); (iii) an amount of alkanediol which increases the antimicrobial effect, for example at a concentration between about 0.3 and 1.0 percent (weight/weight), and (iv) one or more fruit acid at a total concentration between about 0.125 and 2.0 percent (weight/weight).

The preferred essential oils are the ones that show significant enhancement of antimicrobial activity in combination with citric acid. These oils include one or more selected from lemongrass oil, cinnamon oil, basil oil and citronella oil (preferably at a total concentration of between about 0.2 and 0.7 percent (weight/weight), with the optional further addition of orange oil to reduce the pungent odor of the other essential oils and to provide a fragrance which is mild and pleasant. Fruit acids which may be used in such compositions include citric acid or lactic acid (preferably citric acid) at a concentration between about 0.5 and 1.0 percent (weight/weight).

Preferred but non-limiting examples of non-alkanediol alcohols for solubilisation of both essential oils and citric acid are aliphatic alcohols having carbon atoms about 1 to 8 such as methanol, ethanol, n-propanol, isopropyl alcohol, 2-methyl-2 propanol, hexanol, or combinations thereof, at a concentration of between about 5 and 20 percent (weight/weight). Aromatic alcohols, for example, but not by way of limitation, phenoxyethanol, benzyl alcohol, 1-phenoxy-2propanol, and/or phenethyl alcohol, for example at a concentration of between about 0.5 and 5 percent (weight/weight) may also optionally be used in combination with aliphatic alcohols. A further solvent which optionally may be comprised in a composition of the invention is isopropyl myristate. Most preferred aliphatic alcohols include ethanol, denatured alcohol (SDA 40B and SDA 3C) and isopropanol. Most preferred aromatic alcohols include phenoxyethanol and phenethanol.

Compositions comprising lemongrass or cinnamon oils (0.2-0.5% (weight/weight)) and orange oil (0.1-0.2% (weight/weight)), exhibit a pleasant and mild fragrance. Furthermore these oils even at these lower concentrations have been observed to provide superior antibacterial activity (more than 3 log reduction when challenged with 108 colony forming unit of a gram positive pathogen (S. aureus) in combination with a secondary alcohol (0.3-1.0% (weight/weight)) and alcohol (5-20% (weight/weight)).

In specific, non-limiting embodiments, the present invention provides for a skin or surface disinfectant composition comprising the essential oil lemongrass (0.3-0.5% (weight/weight)), orange oil (0.1-0.2% (weight/weight)), citric acid (0.5-2.0% (weight/weight)), SDA 40B alcohol (5-20% (weight/weight)) and 1,2 decanediol (0.3-1.0% (weight/weight)).

Preferably the pH of personal care products is between about 3.5-5.0, and preferably between about 4-4.7.

In addition to the above ingredients, a composition of the invention may optionally further comprise an emollient to further reduce irritation, such as, but not limited to, a fatty alcohol, behentrimonium methosulfate-cetyl alcohol (Incroquat TMS), or a polyol such as glycerol, propylene glycol, diglycerol, ethylene glycol, diethylene glycol, triethylene glycol, dipropylene glycol, tripropylene glycol, hexylene glycol, butylene glycol, etc.

Essential oils are volatile and therefore it is desirable that the antimicrobial composition containing essential oils is incorporated in a suitable base in which it is stable at higher temperature and over a long period of time. Accordingly, a composition of the invention may optionally comprise a hydrophilic or hydrophobic gel forming polymer, a fatty acids, a plant oils etc. Suitable hydrophilic gel polymers include, but are not limited to, hydroxypropylmethyl cellulose, cationic hydroxyethyl cellulose (U-care polymers), ethyl cellulose, hydroxypropyl cellulose, hydroxymethyl cellulose, carboxy methyl cellulose, polyethylene oxide (polyox resins), and chitosan pyrrolidone carboxylate (Kytamer PC), silica gel, carbomerpolymers etc. Suitable hydrophobic gel polymers include, but are not limited to, silicone polymers, for example polydimethylsiloxane polymer (Dow Corning 225 Silicone Fluid), dimethiconol fluid in dimethicone (Dow Corning 1403 Silicone Fluid), cyclomethicone and dimethicone copolyl (Dow Corning 3225C and Q2-5220 Silicone Fluid), silicone glycol (BASF 1066 DCG polyol), KSG series Silicone gels (Shin-etsu), and combinations thereof. Suitable plant oils include, but are not limited to, olive oil, almond oil, avocado oil, basil oil, primrose oil, peanut oil, safflower oil, sesame oil, soyabean oil, wheat germ oil.

### 4.8 Compositions Comprising Essential Oils/ICS, Botanical Extracts, Fruit Acids, and Alkanediols

In non-limiting embodiments, the present invention provides for compositions comprising a low concentration essential oil or IC and a low concentration botanical extract in synergistic combination with a fruit acid and alkanediol. In various non-limiting embodiments, the compositions of the present invention include total stock solution concentrations of essential oils and botanical extracts in concentrations ranging from about 0.5% to about 30%, more preferably from about 2% to about 20%.

In particular, non-limiting embodiments of the invention, stock solutions containing the compositions comprise from about 0.3% to about 15% (w/w), preferably from about 0.5% to about 6.0% (w/w) of essential oils or ICs; from about 0.3% to about 30%, preferably from about 1% to about 20% (w/w), more preferably from about 1% to about 15% (w/w) of botanical extracts; from about 5% to about 20% (w/w), more preferably from about 10% to about 20% (w/w) fruit acids; from about 1% to about 80% (w/w), preferably from about 20% to about 80% (w/w) alkanediols, more preferably from about 30% to about 80% (w/w), more preferably from about 30% to about 50% (w/w) alkanediols; from about 0% to about 90% (w/w), preferably from about 0% to about 80% (w/w) solvents. In other specific non-limiting embodiments, the synergistic compositions of the present invention in final products contain low concentrations of essential oils or ICs ranging from about 0.01% to about 0.5%, more preferably from about 0.02% to about 0.1%; and botanical extracts ranging from about 0.1 to about 0.5%. Solvent systems include, but are not limited to, alcohol, glycerol, diglycerol, propylene glycol, dipropylene glycol, and vegetable oils.

The low concentration compositions of the invention may be used in concentrations from about 1% to about 5% in personal care products or topical creams. Alternatively, from about 10% to about 20% of the stock solutions of the compositions may be used in soap formulations. Additionally, the compositions contain no or little to very mild fragrance.

The compositions of the invention may be used as alternatives to conventional disinfectants or may be combined with to other antimicrobial agents to enhance their activity, particularly providing persistent antimicrobial protection without causing skin sensitivity.

### 4.9 Personal Care Products

In non-limiting embodiments, the present invention provides for personal care product compositions comprising low concentrations of one or more essential oil and/or IC and botanical extracts including plant and fruit extracts, in synergistic combination with one or more fruit acids and alkanediols, as set forth in section 4.8 above. In preferred, non-limiting embodiments, the low concentrations of the active agents are such that regular exposure of skin to the personal care product does not produce skin irritation in a normal subject.

Non-limiting examples of personal care products which may utilize the invention include bar soap, liquid soap (e.g., hand soap), hand sanitizer, cleansing wipes, disinfecting wipes, body wash, acne treatment products, shampoo, conditioner, cosmetics (including but not limited to liquid or powder foundation, liquid or solid eyeliner, mascara, cream eye shadow, tinted powder, “pancake” type powder to be used dry or moistened, etc.) deodorant, antimicrobial creams, body lotion, hand cream, topical cream, aftershave lotion, skin toner, mouth wash, toothpaste, sunscreen lotion, and baby products such as, but not limited to, cleansing wipes, baby shampoo, baby soap, and diaper cream. The present invention may also be applied to wound care items, such as, but not limited to, wound healing ointments, wound coverings, bandages, tape, and steri-strips, and medical articles such as medical gowns, caps, face masks, and shoe-covers, surgical drops, etc.

Personal care compositions according to the invention, in addition to one or more essential oil and/or IC together with one or more fruit acid, may further comprise one or (preferably) more than one component selected from the group consisting of emollients, stabilizing agents, thickening agents, humectants, anti-inflammatory agents, antimicrobial agents, neutralizing agents, surfactants, water, silicone polymers, alcohols, and hydrogels, as well as additional components as may be known in the art. Non-limiting examples of such components are set forth below.

In various non-limiting embodiments of the invention, a personal care product comprising a combination of one or more essential oil and/or IC together with one or more fruit acid may further comprise an emollient, for example PEG 20 almond glycerides, Probutyl DB-10, Glucam P-20, Glucam E-10, Glucam P-10, Glucam E-20, Glucam P-20 distearate, glycerin, propylene glycol, octoxyglycerine, cetyl acetate, acetylated lanolin alcohol (e.g., Acetulan), cetyl ether (e.g., PPG-10), myristyril ether (e.g., PPG-3), hydroxylated milk glycerides (e.g., Cremeral HMG), polyquaternium compounds (e.g., U-care compounds), copolymers of dimethyl dialyl ammonium chloride and acrylic acid (e.g., Merquat), dipropylene glycol methyl ethers (e.g., Dowanol DPM, Dow Corning), polypropylene glycol ethers (e.g., Ucon 50-HB-600, Union Carbide) and silicon polymers. Other suitable emollients may include hydrocarbon-based emollients such as petrolatum or mineral oil, fatty ester-based emollients, such as methyl, isopropyl and butyl esters of fatty acids such as isopropyl palmitate, isopropyl myristate, isopropyl isostearate, isostearyl isostearate, diisopropyl sebacate, and propylene dipelargonate, 2-ethylhexyl isononoate, 2-ethylhexyl stearate, C₁₂-C₁₆ fatty alcohol lactates such as cetyl lactate and lauryl lactate, isopropyl lanolate, 2-ethylhexyl salicylate, cetyl myristate, oleyl myristate, oleyl stearate, oleyl oleate, hexyl laurate, and isohexyl laurate. Additional useful emollients include lanolin, olive oil, cocoa butter, and shea butter.

In various non-limiting embodiments of the invention, a personal care product comprising a combination of one or more essential oil and/or IC together with one or more fruit acid may further comprise a stabilizing agent consisting of antioxidants, including but not limited to vitamin C (ascorbic acid) and vitamin E (tocopherol), and surfactants, including but not limited to incromide or silicone-based surfactants (Masil SF-19, BASF).

In various non-limiting embodiments of the invention, a personal care product comprising a combination of one or more essential oil and/or IC together with one or more fruit acid may further comprise a thickening and/or gelling agent such as stearyl alcohol, cationic hydroxy ethyl cellulose (Ucare; JR30), hydroxy propyl methyl cellulose, hydroxy propyl cellulose (Klucel), chitosan pyrrolidone carboxylate (Kytamer), behenyl alcohol, zinc stearate, emulsifying waxes, including but not limited to Incroquat and Polawax, an addition polymer of acrylic acid, a resin such as Carbopol® ETD™ 2020, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminium silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentotnite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof. The gelling agents used in vehicles may be natural gelling agents such as natural gums, starches, pectins, agar and gelatin. Often, the gelling agents are based on polysaccharides or proteins Examples include but are not limited to guar gum, Xanthum gum, Alginic acid (E400), sodium alginate (E401), potassium alginate (E402), ammonium alginate (E403), calcium alginate (E404, -polysaccharides from brown algae), Agar (E406, a polysaccharide obtained from red seaweeds), Carrageenan (E407, a polysaccharide obtained from red seaweeds), Locust bean gum (E410, a natural gum from the seeds of the Carob tree), Pectin (E440, a polysaccharide obtained from apple or citrus-fruit), and Gelatin (E441, made by partial hydrolysis of animal collagen).

In various non-limiting embodiments of the invention, a personal care product comprising a combination of one or more essential oil and/or IC together with one or more fruit acid may further comprise a humectant, such as, for example, glycerin, 1-2-propylene glycol, dipropylene glycol, polyethylene glycol, 1,3-butylene glycol, or 1,2,6-hexanetriol.

In certain non-limiting embodiments of the invention, essentially the entire antimicrobial effect of the inventive composition is achieved by an antimicrobial composition consisting of one or more essential oil and/or one or more IC, together with a fruit acid and optionally an alcohol. In alternative embodiments of the invention, one or more additional antimicrobial agent may be comprised, for example, in the amount of between about 0.05 and 2.0 percent (weight/weight), where such antimicrobial agent may be selected from the group consisting of iodophors, iodine, benzoic acid, dihydroacetic acid, propionic acid, sorbic acid, methyl paraben, ethyl paraben, propyl paraben, butyl paraben, cetrimide, benzalkonium chloride, dequalinium chloride, chlorhexidine, chloroeresol, chlorxylenol, benzyl alcohol, bronopol, chlorbutanol, phenoxyethanol, phenylethyl alcohol, 2,4-dichlorobenzyl alcohol, thiomersal, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, silver sulfadiazine, octoxyglycerine, biguanides such as, but not limited to, chlorhexidine free base, chlorhexidine palmitate, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-a-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynapthoate, chlorhexidine embonate, and parahexamethylenebiguanide (“PHMB”).

In various non-limiting embodiments of the invention, a personal care product comprising a combination of one or more essential oil and/or IC together with one or more fruit acid may further comprise a neutralizing agent to neutralize carboxyl groups present in one or more other component, such as carboxyl groups in a thickening agent. Suitable neutralizing agents include diisopropylamine and triethanolamine.

In various non-limiting embodiments of the invention, a personal care product comprising a combination of one or more essential oil and/or IC together with one or more fruit acid may further comprise a surfactant. The surfactant may be an anionic surfactant, a cationic surfactant, an ampholytic surfactant, or a nonionic surfactant. Examples of nonionic surfactants include polyethoxylates, fatty alcohols (e.g., ceteth-20 (a cetyl ether of polyethylene oxide having an average of about 20 ethylene oxide units) and other “BRIJ”® nonionic surfactants available from ICI Americas, Inc. (Wilmington, Del.)), cocamidopropyl betaine, alkyl phenols, fatty acid esters of sorbitol, sorbitan, or polyoxyethylene sorbitan. Suitable anionic surfactants include ammonium lauryl sulfate and lauryl ether sulfosuccinate. A preferred surfactant is lauroyl ethylenediamine triacetic acid sodium salt at a concentration between about 0.5-2.0% (weight/weight). In particular non-limiting embodiments of the invention, concentrations of surfactant are between about 0.05% and 2% (weight/weight).

In various non-limiting embodiments of the invention, a personal care product may comprise water.

In various non-limiting embodiments of the invention, a personal care product comprising a combination of one or more essential oil and/or IC together with one or more fruit acid may further comprise a hydrogel comprising, for example, a compound such as hydroxypropylmethyl cellulose, cationic hydroxyethyl cellulose (U-care polymers), ethyl cellulose, hydroxypropyl cellulose, hydroxymethyl cellulose, carboxy methyl cellulose, polyethylene oxide (polyox resins), and chitosan pyrrolidone carboxylate (Kytomer PC).

In various non-limiting embodiments of the invention, a personal care product comprising a combination of one or more essential oil and/or IC together with one or more fruit acid may further comprise an alcohol or a mixture of alcohols, for example, ethanol, isopropyl alcohol, n-propyl alcohol, and mixtures thereof; fatty alcohols, including, but not limited to, cetyl alcohol, myristol alcohol, stearyl alcohol, octyl alcohol, decyl alcohol and lauryl alcohol, and mixtures thereof; and hexanol.

In various non-limiting embodiments of the invention, a personal care product comprising a combination of one or more essential oil and/or IC together with one or more fruit acid may further comprise a silicone polymer, for example one or more than one polydimethylsiloxane polymer (Dow Corning 225 Silicone Fluid), dimethiconol fluid in dimethicone (Dow Corning 1403 Silicone Fluid), cyclomethicone and dimethicone copolyl (Dow Corning 3225C Silicone Fluid), and silicone glycol (BASF 1066 DCG polyol). In particular, non-limiting embodiments, the amount of silicone polymer is between about 0.1 and 1.0 percent (volume/volume).

In various non-limiting embodiments of the invention, a personal care product comprising a combination of one or more essential oil and/or IC together with one or more fruit acid may further comprise an emollient solvent such as a glycidyl ether having an alkyl chain up to and including 18 carbon molecules and ethoxylates and propoxylates thereof, a glyceryl ether having an alkyl chain up to and including 18 carbon molecules and ethoxylates and propoxylates thereof, a mono- or diglyceryl ether having an alkyl chain up to and including 18 carbon molecules and ethoxylates and propoxylates thereof, ethoxylate and propoxylate ethers, ethoxy diglycol esters, ethyl hexyl alcohol propoxylate, and propylene glycol esther ethoxylates and propoxylates, and Arlamol (Altas).

In various non-limiting embodiments of the invention, a personal care product comprising a combination of one or more essential oil and/or IC together with one or more fruit acid may further comprise additives such as dyes, fragrances, pH adjusters, including basic pH adjusters such as ammonia, mono-, di- and tri alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine); acid pH adjusters such as mineral acids and polycarboxylic acids (e.g., hydrochloric acid, nitric acid, phosphoric acid, sulfuric acid, citric acid, glycolic acid, and lactic acid); vitamins such as vitamin A, vitamin E and vitamin C; polyamino acids and salts, such as ethylenediamine tetraacidic acid (EDTA), preservatives such as Germall plus and DMDM hydantoin, and sunscreens such as aminobenzoic acid, arobenzone, cinoxate, diioxybenzone, homosalate, menthyl anthranilate, octocrylene, octyl methoxycinnamate, octyl salicylate, oxybenzoate, padimate O, phenylbenzimidazole, sulfonic acid, sulisobenzone, titanium dioxide, trolamine salicylate and zinc oxide.

In one set of non-limiting embodiments, the present invention provides for personal care compositions comprising one or more EO/IC, preferably where the EO(s)/IC(s) are selected from the group consisting of lemongrass oil and/or an IC thereof, orange oil and/or an IC thereof, cinnamon leaf oil and/or an IC thereof, basil oil and/or an IC thereof, eugenol, cinnamaldehyde, cinnamylacetic ester, and cinnamic acid, at a total concentration of between about 0.1 and 1% (weight/weight); a fruit acid, preferably citric acid, at a concentration of between about 0.125 and 1% (weight/weight); an alcohol, preferably ethanol, at a concentration of between about 5 and 20% (weight/weight); and optionally triclosan at a concentration of between about 0.05 and 1% (weight/weight), where the ratio of EO(s)/IC(s) to the fruit acid(s) is between about 1:1 to 1:10 and the pH is between about 3 and about 7, preferably between about 5 and 6.

In another set of non-limiting embodiments, the present invention provides for personal care compositions comprising lemongrass oil or an IC thereof and orange oil or an IC thereof at a total concentration of between about 0.2 and 0.7% (weight/weight); a fruit acid, preferably citric acid, at a concentration of between about 0.25 and 1% (weight/weight); an alcohol, preferably ethanol, at a concentration of between about 5 and 20% (weight/weight); and optionally triclosan at a concentration of between about 0.05 and 1% (weight/weight), where the ratio of EO(s)/IC(s) to fruit acid(s) is between about 1:1 to 1:5 and the pH is between about 3 and about 7, preferably between 5 and 6.

In various non-limiting embodiments of the invention, a personal care product comprising a combination of one or more essential oil and/or IC together with one or more fruit acid may further comprise various anti-inflammatory, antimicrobial agents, anti-irritants, and gelling ingredients. Such compositions may be included in, for example, wound healing ointments. The antimicrobial botanicals contemplated for wound treatment include 0.2-0.7% (weight/weight) essential oils such as lemongrass oil (LG) or orange oil (O), and 0.2-1.0% (weight/weight) fruit acids such as citric acid (Cit) and lactic acid (L), and 0.5-1.0% (weight/weight) phenoxyethanol, which is a constituent of sage oil (PXE). Anti-irritant, anti inflammatory botanicals include, but are not limited to 0.3-0.7% (weight/weight) Calendula oil (Co), 0.1-0.5% (weight/weight) turmeric extract (curcumin (Cr)), 0.2-2.0% (weight/weight) salicylic acid (S), 0.2-0.5% (weight/weight) Camphor (Cm) and 2-30% (weight/weight) honey (H). Gelling agents would include, but are not limited to, Guar gum, Xanthum gum Alginic acid, and Pectin in amounts of 0.2-3.0% (weight/weight).

In one specific, non-limiting embodiment, the present invention provides for a liquid soap product called “CN1-A” having one of the following compositions (CN1-A1 OR CN1-A2)

TABLE 1
CN1-A Compositions

| | Ingredient | % (weight/weight) |
|---|---|---|
| CN1-A1: | Deionized water | 59.15 |
| | Polyox N 60K | 0.2 |
| | Pluronic F 87 Prill | 2.0 |
| | Ucare Jr 30 | 0.4 |
| | D,L Panthenol 50 W | 1.0 |
| | Incromide oxide L | 3.0 |
| | Crosultane C-50 | 3.0 |
| | Montalene C 40 | 3.0 |
| | 2-Phenoxy-ethanol | 1.0 |
| | Zinc gluconate | 0.1 |
| | Glycerine | 2.0 |
| | SDA-40B alcohol | 15.5 |
| | Cinnamon leaf oil | 0.5 |
| | Citric acid | 1.0 |
| | Orange oil | 0.2 |
| | Distilled water | 7.95 |
| CN1-A2: | Deionized water | 59.25 |
| | Polyox N 60K | 0.2 |
| | Pluronic F 87 Prill | 2.0 |
| | Ucare Jr 30 | 0.4 |
| | D,L Panthenol 50 W | 1.0 |
| | Incromide oxide L | 3.0 |
| | Crosultane C-50 | 3.0 |
| | Montalene C 40 | 3.0 |
| | 2-Phenoxy-ethanol | 1.0 |
| | Glycerine | 2.0 |
| | SDA-40B alcohol | 15.5 |
| | Cinnamon leaf oil | 0.5 |
| | Citric acid | 1.0 |
| | Orange oil | 0.2 |
| | Distilled water | 7.95 |

In another specific, non-limiting embodiment, the present invention provides for a liquid soap product called “CN1-B” having the following composition.

TABLE 2
CN1-B

| Ingredient | % (weight/weight) |
|---|---|
| Deionized water | 63.2 |
| Methocel 40-101 | 0.1 |
| Pluronic F 87 Prill | 0.1 |
| Ucare Jr 30 | 0.1 |
| D,L Panthenol 50 W | 1.0 |
| Incromide oxide L | 3.0 |
| Crosultane C-50 | 3.0 |
| Montalene C 40 | 1.5 |
| 2-Phenoxy-ethanol | 1.0 |
| Glycerine | 2.0 |
| SDA-40B alcohol | 15.5 |
| Cinnamon leaf oil | 0.5 |
| Citric acid | 1.0 |
| Orange oil | 0.2 |
| Distilled water | 7.8 |

In another specific, non-limiting embodiment, the present invention provides for a liquid soap product called “CN1-C” having the following composition.

TABLE 3
CN1-C

| Ingredient | % (weight/weight) |
|---|---|
| Deionized water | 63.2 |
| Methocel 40-101 | 0.1 |
| Pluronic F 87 Prill | 0.1 |
| Ucare Jr 30 | 0.1 |
| D,L Panthenol 50 W | 1.0 |
| Incromide oxide L | 3.0 |
| Crosultane C-50 | 3.0 |
| Montalene C 40 | 1.5 |
| 2-Phenyl-ethanol | 1.0 |
| Glycerine | 2.0 |
| SDA-40B alcohol | 15.5 |
| Cinnamon leaf oil | 0.5 |
| Citric acid | 1.0 |
| Orange oil | 0.2 |
| Distilled water | 7.8 |

In a subset of non-limiting embodiments, the present invention provides for a soap comprising one or more essential oil, 1% citric acid, and a soap base comprising a surfactant, an emollient, and a thickener, and having a pH between about 3-5. Specific non-limiting examples of such soaps follow.

TABLE 4
Soap Containing Lemongrass oil, and Citric acid (LG-Cit-4)
(4 represents total oil 0.4%)

| Ingredient | % (w/w) |
|---|---|
| Deionized Water | 63.5 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Lemongrass oil | 0.4 |

TABLE 5
Soap Containing Lemongrass oil, and Citric acid (LG-Cit-6)
(6 represents total oil 0.6%)

| Ingredient | % (w/w) |
|---|---|
| Deionized Water | 63.3 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Lemongrass oil | 0.6 |

TABLE 6
Soap Containing Lemongrass oil, Orange oil (O oil) and
Citric acid (LGO-Cit 6) (6 represents total oil 0.6%)

| Ingredient | % (w/w) |
|---|---|
| Deionized Water | 63.3 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Lemongrass oil | 0.4 |
| Citric acid | 1.0 |
| Orange oil | 0.2 |

TABLE 7
Soap Containing Lemon grass oil, Orange oil and
Citric acid (LGO-Cit 7) (7 represents total oil 0.7%)

| Ingredient | % (w/w) |
|---|---|
| Deionized Water | 63.2 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Lemongrass oil | 0.5 |
| Citric acid | 1.0 |
| Orange oil | 0.2 |

TABLE 8
Soap Containing Cinnamon oil, Orange oil and Citric
acid (CO-Cit 6) (6 represents total oil 0.6%)

| Ingredient | % (w/w) |
|---|---|
| Deionized Water | 63.3 |
| Methocel 40-101 | 0.1 |
| Pluronic F 87 Prill | 0.1 |
| Ucare Jr 30 | 0.1 |
| DL Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Cinnamon oil | 0.4 |
| Citric acid | 1.0 |
| Orange oil | 0.2 |

TABLE 9
Soap Containing Cinnamon oil, Orange oil and Citric acid
(CO-Cit 7) (7 represent total oil 0.7%)

| Ingredient | % (w/w) |
|---|---|
| Deionized Water | 63.2 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Cinnamon oil | 0.5 |
| Citric acid | 1.0 |
| Orange oil | 0.2 |

TABLE 10
Soap Containing Orange oil and Citric acid (O-Cit 2)
(2 represents total oil 0.2%)

| Ingredient | % (w/w) |
|---|---|
| Deionized Water | 63.7 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Orange oil | 0.2 |
| Citric acid | 1.0 |

TABLE 11
Soap Containing Basil oil (“B oil”), Orange oil (“O oil”)
and Citric acid (BO-Cit 6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Basil oil | 0.4 |
| Citric acid | 1.0 |
| Orange oil | 0.2 |

TABLE 12
Soap containing Citronella oil (“CR oil”), Orange oil(“O oil”),
Citric acid (CRO-Cit6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Citronella oil | 0.4 |
| Citric acid | 1.0 |
| Orange oil | 0.2 |

In further specific, non-limiting embodiments, the present invention provides for the following combinations of agents in a soap base (percentages weight/weight):
0.15% TC+0.4% lemongrass oil+0.2% orange oil+1% citric acid;
0.4% lemongrass oil+0.2% orange oil+1% citric acid; or
0.15% TC+0.4% cinnamon oil+0.2% orange oil+1% citric acid; or
0.4% cinnamon oil+0.2% orange oil+1% citric acid.

In still further specific, non-limiting embodiments, the present invention provides for the following combinations of agents in a soap base (percentages w/w):
Cinnamon oil 0.5%+Orange Oil 0.2%+Citric acid 1.0%+alcohol (e.g., denatured ethyl alcohol, such as SDA 40 B) 5.5%+TC 0.14% (or TC 0.15%); or
Lemongrass oil 0.5%+Orange Oil 0.2%+Citric acid 1.0%+alcohol (e.g., denatured ethyl alcohol, such as SDA 40B) 5.5%+TC 0.14% (or TC 0.15%); or
Lemongrass oil 0.5%+Citric acid 1.0%+alcohol (e.g. denatured ethyl alcohol such as SDA 40 B)5.5%+TC 0.14% (or TC 0.15%).

In specific non-limiting embodiments, the present invention provides for compositions comprising (0.2-0.3% (weight/weight)) of essential oils such as lemongrass or cinnamon and 0.1-0.2% (weight/weight) orange oil when used in combination with 1% citric acid and alkanediols such as 1,2 decanediol, 1,2 dodecanediol and 1,12 dodecanediol, as set forth above. In a specific, non-limiting embodiment, the present invention provides for a soap formulation comprising 0.3% (weight/weight) of lemongrass oil or cinnamon oil in combination with 0.1% (weight/weight) orange oil, and 1% (weight/weight) citric acid with and without alkanediols, where the pH preferably is between 4.5-4.6. The following Tables provide non-limiting embodiments of the present invention.

TABLE 13
Soap containing Lemon grass oil, Orange oil and Citric acid
(LG-O-Cit 5) (5 represents total oil 0.5%)

| Ingredient | Percentage (w/w) |
|---|---|
| Deionized Water | 64.8 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.2 |
| pH 4.55 | |

TABLE 14
Soap Containing Lemon grass oil, Orange oil and Citric acid
(LG-O-Cit 4) (4 represents total oil 0.4%)

| Ingredient | Percentage (w/w) |
|---|---|
| Deionized Water | 64.9 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| pH 4.64 | |

TABLE 15
Soap Containing LG-O-Cit 5 and 0.3% 1,2 Decanediol

| Ingredient | Percentage (w/w) |
|---|---|
| Deionized Water | 64.5 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.2 |
| 1,2 Decanediol | 0.3 |
| pH 4.6 | |

TABLE 16
Soap Containing LG-O-Cit-4 and 0.3% 1,2 Decanediol

| Ingredient | Percentage (w/w) |
|---|---|
| Deionized Water | 64.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,2 Decanediol | 0.3 |
| pH 4.6 | |

TABLE 17
Soap Containing LG-O-Cit 4, 0.3% 1,2 Decanediol + 0.5%
Incroquat behenyl TMS

| Ingredient | Percentage (w/w) |
|---|---|
| Deionized Water | 64.1 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,2 Decanediol | 0.3 |
| Incroquat TMS | 0.5 |
| pH 4.6 | |

TABLE 18
Soap Containing LG-O-Cit 4 and 0.3% 1,2 Dodecanediol

| Ingredient | Percentage (w/w) |
|---|---|
| Deionized Water | 64.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,2 Decanediol | 0.3 |
| pH 4.6 | |

TABLE 19
Soap Containing LG-O-Cit 4 and 0.3% 1,12 Dodecanediol

| Ingredient | Percentage (w/w) |
|---|---|
| Deionized Water | 64.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,12 Dodecanediol | 0.3 |
| pH 4.6 | |

TABLE 20
Soap Containing LG-O-Cit 4 and 0.3% 1,2 tetradecanediol

| Ingredient | Percentage (w/w) |
|---|---|
| Deionized Water | 64.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,2 Tetradecanediol | 0.3 |
| pH 4.6 | |

TABLE 21
Soap Containing LG-O-Cit 4A
(Same as LG-O-Cit 4 but contains
17% SDA-40B alcohol instead of 15%)

| Ingredient | Percentage (w/w) |
|---|---|
| Deionized Water | 62.9 |
| Methocel 40-101 | 0.2 |
| Pluronic F 87 Prill | 1.0 |
| Polyox WSR- N- 60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemongrass oil | 0.3 |
| Citric acid | 1.0 |
| Orange oil | 0.1 |
| pH 4.64 | |

TABLE 22
Soap Containing LG-O-Cit 4A and 0.5% 1,2 Decanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR- N- 60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemongrass oil | 0.3 |
| Citric acid | 1.0 |
| Orange oil | 0.1 |
| 1,2 Decanediol | 0.5 |
| pH 4.6 | |

TABLE 23
Soap Containing LG-O-Cit 4A and 0.5% 1,2 Dodecanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR- N- 60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemongrass oil | 0.3 |
| Citric acid | 1.0 |
| Orange oil | 0.1 |
| 1,2 Dodecanediol | 0.5 |
| pH 4.6 | |

TABLE 24
Soap Containing LG-O-Cit 4A and 0.5% 1,12 Dodecanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR- N- 60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemongrass oil | 0.3 |
| Citric acid | 1.0 |
| Orange oil | 0.1 |
| 1,12 Dodecanediol | 0.5 |
| pH 4.6 | |

TABLE 25
Soap Containing LG-O-Cit 4A and 0.5% 1,2 Tetradecanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR- N- 60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemongrass oil | 0.3 |
| Citric acid | 1.0 |
| Orange oil | 0.1 |
| 1,2 tetradecanediol | 0.5 |
| pH 4.6 | |

TABLE 26
Soap Containing Cn-O-Cit 4A

| Ingredient | Percentage (w/w) |
|---|---|
| Deionized Water | 62.9 |
| Methocel 40-101 | 0.2 |
| Pluronic F 87 Prill | 1.0 |
| Polyox WSR- N- 60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Cinnamon oil | 0.3 |
| Citric acid | 1.0 |
| Orange oil | 0.1 |
| pH 4.64 | |

TABLE 27
Soap Containing Cn-O-Cit 4A + 0.5% 1,2 decanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR- N- 60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Cinnamon oil | 0.3 |
| Citric acid | 1.0 |
| Orange oil | 0.1 |
| 1,2 Decanediol | 0.5 |
| pH 4.64 | |

TABLE 28
Soap Base

| Ingredient | Percentage (w/w) |
|---|---|
| Deionized Water | 81.3 |
| Methocel 40-101 | 0.2 |
| Pluronic F 87 Prill | 1.0 |
| Polyox WSR- N- 60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| pH 4.64 (adjusted with 10N | |
| hydrochloric acid) | |

In certain non-limiting embodiments of the invention, where the compositions are used in soap formulations, the compositions may contain from about 0.5% to about 55% (w/w), preferably from about 0.5% to about 15% (w/w) essential oils or ICs; from about 1% to about 30% (w/w), preferably from about 1% to about 5% (w/w) botanical extracts; from about 1% to about 20% (w/w), preferably from about 1% to about 10% (w/w) alkanediols; from about 5% to about 20% (w/w), preferably from about 5% to about 15% (w/w) fruit acids; and from about 0% to about 90% (w/w) solvents. The following tables provide non-limiting embodiments of the present invention that contain a soap base.

TABLE 29
LG-6-O

| Ingredient | % (w/w) |
|---|---|
| Lemongrass oil | 0.3 |
| Grapefruit seed extract | 0.2 |
| Orange oil | 0.1 |
| Octanediol | 0.5 |
| SDA 3C | 4.9 |
| Citric Acid | 1.0 |
| Softsoap Base (Colgate | 93.0 |
| Palmolive) | |

TABLE 30
LG-6-S

| Ingredient | % (w/w) |
|---|---|
| Lemongrass oil | 0.3 |
| Grapefruit seed extract | 0.2 |
| Orange oil | 0.1 |
| Symclariol | 0.5 |
| Phenoxyethanol | 1.0 |
| SDA 3C | 3.9 |
| Citric Acid | 1.0 |
| Softsoap Base (Colgate | 93.0 |
| Palmolive) | |

TABLE 31
LG-6-O-TC

| Ingredient | % (w/w) |
|---|---|
| Lemongrass oil | 0.3 |
| Grapefruit seed extract | 0.2 |
| Orange oil | 0.1 |
| Octanediol | 0.5 |
| SDA 3C | 4.75 |
| Citric Acid | 1.0 |
| Triclosan | 0.15 |
| Softsoap Base (Colgate | 93.0 |
| Palmolive) | |

TABLE 32
LG-19-O

| Ingredient | % (w/w) |
|---|---|
| Water | 63.1 |
| Methocil (40-101) | 0.2 |
| U-care Jr | 0.3 |
| Pluronic F-87 | 1.0 |
| Montalene C-40 | 2.0 |
| Incromine oxide L | 8.0 |
| Crosultane C-50 | 3.0 |
| Glycerine | 2.0 |
| Polyoxyl SR-N-60K | 0.2 |
| SDA 40 B | 17.1 |
| Citric acid | 1.0 |
| Lemongrass oil | 0.3 |
| Orange oil | 0.1 |
| Grapefruit seed extract | 0.2 |
| Phenoxy ethanol | 1.0 |
| Octanediol | 0.5 |

TABLE 33
LG-19-S

| Ingredient | % (w/w) |
|---|---|
| Water | 63.1 |
| Methocil (40-101) | 0.2 |
| U-care Jr | 0.3 |
| Pluronic F-87 | 1.0 |
| Montalene C-40 | 2.0 |
| Incromine oxide L | 8.0 |
| Crosultane C-50 | 3.0 |
| Glycerine | 2.0 |
| Polyoxyl SR-N-60K | 0.2 |
| SDA 40 B | 17.1 |
| Citric acid | 1.0 |
| Lemongrass oil | 0.3 |
| Orange oil | 0.1 |
| Grapefruit seed extract | 0.2 |
| Phenoxy ethanol | 1.0 |
| Symclariol | 0.5 |

The following Tables provide non-limiting lotion embodiments of the present invention.

TABLE 34
Antibacterial topical lotion comprising
LG-O-Cit A + 1,2Decanediol (“LG-O-Cit A-D Lotion”)

| Ingredient | Percentage (w/w) |
|---|---|
| Water | 65.9 |
| U Care-JR 30M | 0.25 |
| PolyoxWSR-205 | 0.1 |
| Incroquat TMS Behenyl | 2.0 |
| Isopropyl myristate | 1.0 |
| Acetulan | 1.0 |
| Vitamin E | 0.2 |
| Zinc stearate | 0.2 |
| Polawax NF | 2.75 |
| Glycerin | 2.0 |
| Allantoin | 0.2 |
| Dimethicone copolyol (Q2-5220) | 2.5 |
| Citric acid | 1.0 |
| 1,2 decanediol | 0.5 |
| Tocopheryl acetate | 0.5 |
| Glyceryl stearate (Arlacel165) | 1.0 |
| Butylene glycol | 3.0 |
| SDA-40-B | 15 |
| Lemongrass oil | 0.5 |
| Tea tree oil | 0.5 |
| Orange oil | 0.1 |
| 1,2 Decanediol(Symclairol) | 0.5 |
| (pH adjusted to 4.5-5.0) | |

TABLE 35
Antibacterial topical lotion comprising
LG-O-Cit A + 1,2Decanediol + Triclosan
(“LG-O-Cit A-D-T Lotion”)

| Ingredient | Percentage (w/w) |
|---|---|
| Water | 65.6 |
| U Care-JR 30M | 0.25 |
| PolyoxWSR-205 | 0.1 |
| Incroquat TMS Behenyl | 2.0 |
| Isopropyl myristate | 1.0 |
| Acetulan | 1.0 |
| Vitamin E | 0.2 |
| Zinc stearate | 0.2 |
| Polawax NF | 2.75 |
| Glycerin | 2.0 |
| Allantoin | 0.2 |
| Dimethicone copolyol (Q2- | 2.5 |
| 5220) | |
| Citric acid | 1.0 |
| 1,2 decanediol | 0.5 |
| Tocopheryl acetate | 0.5 |
| Glyceryl stearate (Arlacel165) | 1.0 |
| Butylene glycol | 3.0 |
| SDA-40-B | 15 |
| Lemongrass oil | 0.5 |
| Tea tree oil | 0.5 |
| Orange oil | 0.1 |
| 1,2 Decanediol(Symclairol) | 0.5 |
| Triclosan | 0.3 |
| (pH adjusted to 4.5-5.0) | |

TABLE 36
Antibacterial-anti inflammatory topical lotion comprising
LG-O-CitA + 1,2 Decanediol (“LG-O-Cit A-D AB/AIF Lotion”)

| Ingredient | Percentage (w/w) |
|---|---|
| Water | 65.6 |
| U Care-JR 30M | 0.25 |
| PolyoxWSR-205 | 0.1 |
| Incroquat TMS Behenyl | 2.0 |
| Isopropyl myristate | 1.0 |
| Acetulan | 1.0 |
| Vitamin E | 0.2 |
| Zinc stearate | 0.2 |
| Polawax NF | 2.75 |
| Glycerin | 2.0 |
| Allantoin | 0.2 |
| Dimethicone copolyol (Q2-5220) | 2.5 |
| Citric acid | 1.0 |
| 1,2 decanediol | 0.5 |
| Tocopheryl acetate | 0.5 |
| Glyceryl stearate (Arlacel165) | 1.0 |
| Butylene glycol | 3.0 |
| SDA-40-B | 15 |
| Lemongrass oil | 0.5 |
| Tea tree oil | 0.5 |
| Orange oil | 0.1 |
| 1,2 Decanediol(Symclairol) | 0.5 |
| Curcumin | 0.2 |
| Camphor | 0.1 |
| (pH adjusted to 4.5-5.0) | |

Specific non-limiting examples of antimicrobial formulations follow below.

TABLE 37
Antimicrobial Impregnation solution

| Ingredient | Percentage (w/w) |
|---|---|
| Lemongrass oil | 0.3 |
| Orange oil | 0.1 |
| Tea tree oil | 0.5 |
| Calandula oil | 0.5 |
| Citric acid | 1.0 |
| Olive oil | 5.0 |
| Propylene glycol | 10 |
| Decanediol | 0.5 |
| SDA 40 B alcohol | 51.7 |
| U care JR 30 | 0.4 |
| Water | 30 |

TABLE 38
Antimicrobial/anti-inflammatory Impregnation solution

| Ingredient | Percentage (w/w) |
|---|---|
| Lemongrass oil | 0.3 |
| Orange oil | 0.1 |
| Tea tree oil | 0.3 |
| Calandula oil | 0.5 |
| Citric acid | 1.0 |
| Olive oil | 5.0 |
| Propylene glycol | 10 |
| Decanediol | 0.5 |
| SDA 40 B alcohol | 51.0 |
| U care JR 30 | 0.4 |
| Curcumin | 0.3 |
| Water | 29.7 |

In specific, non-limiting embodiments, the present invention provides for the preparation of topical cream formulations containing anti-irritant, anti-inflammatory agents, gelling agents, and botanicals for minor cuts and wounds. Specific cream formulations are as follows.

TABLE 39
Cream 1 (LGO-L-PXE-Co)

| Ingredients | Percentage (w/w) |
|---|---|
| Water | 65.74 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |

pH adjusted to 4.5 with 10 N NaOH

TABLE 40
Cream 2 (LGO-L-PXE-Co-S)

| Ingredients | Percentage (w/w) |
|---|---|
| Water | 65.54 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |
| Salicylic acid | 0.2 |

pH adjusted to 4.5 with 10 N NaOH

TABLE 41
Cream 3 (LGO-L-PXE-Co-Cr)

| Ingredients | Percentage (w/w) |
|---|---|
| Water | 59.25 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |
| Curcumin | 0.2 |

pH adjusted to 4.5 with 10 N NaOH

TABLE 42
Cream 4 (LGO-L-PXE-Co-Cm-H)

| Ingredients | Percentage (w/w) |
|---|---|
| Water | 55.54 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |
| Camphor | 0.2 |
| Honey | 10.0 |

pH adjusted to 4.5 with 10 N NaOH

TABLE 43
Cream 5 (LGO-L-PXE-Co-S)

| Ingredients | Percentage (w/w) |
|---|---|
| Water | 65.04 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |
| Salicylic acid | 0.2 |
| 1,2-decanediol | 0.5 |

pH adjusted to 4.5 with 10 N NaOH

### 4.10 Veterinary Products

In a subset of non-limiting embodiments, the present invention provides for veterinary products comprising a combination of one or more essential oil and/or IC together with one or more fruit acid, as set forth in section 4.4 or 4.5, above. The term “veterinary”, as used here, means “pet care”, and includes home use as well as use in a veterinary office or other pet care establishment.

Non-limiting examples of veterinary care products which may utilize the invention include pet shampoo, pet cleansing wipes including body wipes, ear wipes, and eye wipes, ear cleaning liquid, cage cleaner, surface cleaner for housebreaking accidents, topical creams, ointments, teat dip therapeutic for mastitis and liquid to be applied to pet's skin (as in a “body splash”).

Veterinary care compositions according to the invention, in addition to one or more essential oil and/or IC together with one or more fruit acid, may further comprise one or (preferably) more than one component selected from the group consisting of emollients, stabilizing agents, thickening agents, humectants, antimicrobial agents, neutralizing agents, surfactants, water, silicone polymers, alcohols, and hydrogels, anti-inflammatory agents, wound healing agents, salicylic acid, as well as additional components as may be known in the art.

Specific, non-limiting examples of additional components which may be comprised in pet care products include the components listed above for personal care products.

In certain non-limiting embodiments of the invention, the compositions may be prepared for teat dip to treat mastitis. A general formulation for teat dip compositions is as follows.

TABLE 44
General formulation for teat dip

| Ingredient | Percentage (w/w) |
|---|---|
| anti-irritants | 0.1-5.0% |
| a vehicle containing gelling | 0.2-1.0% |
| agent | |
| glycerin | 5-15% |
| water | 50-80% |
| antimicrobial agents consisting | 0.2-2%), |
| of botanicals | |
| aliphatic and aromatic alcohols | 0.5-15% |
| alkanediol | 0.3-1% |
| solvents such as vegetable oils | |

The anti-irritants used for teat dip may include but are not limited to zinc salts with panthenol, or Bisabolol with ginger root extract (symrelief), or symrelief with a zinc salt. The gelling agents in the vehicle may include but are not limited to natural gelling agents such as natural gums, starches, pectins, agar and gelatin. Antimicrobial botanicals may include but are not limited to lemongrass oil, orange oil and fruit acids such as citric and lactic acid, phenoxyethanol (constituent of sage oil). The following Tables summarize various non limiting examples of formulations.

TABLE 45
Veterinary Composition 1

| Ingredients | Percentage (w/w) |
|---|---|
| Water | 72.14 |
| Xanthum gum | 0.4 |
| Glycerin | 15.0 |
| Zinc gluconate | 0.1 |
| Zinc lactate | 0.1 |
| Zinc acetate | 0.1 |
| Panthenol (75W) | 0.66 |
| Lemon grass oil | 0.3 |
| Orange oil | 0.1 |
| Citric acid | 0.5 |
| Phenoxyethanol | 0.7 |
| Safflower oil | 10.0 |

pH adjusted to 5.0 with 10 N NaOH

TABLE 46
Veterinary Composition 2

| Ingredients | Percentage (w/w) |
|---|---|
| Water | 71.04 |
| Xanthum gum | 0.5 |
| Glycerin | 15.0 |
| Bisabolol, ginger root extract | 0.2 |
| (symrelief) | |
| Panthenol (75W) | 0.66 |
| Lemon grass oil | 0.3 |
| Orange oil | 0.1 |
| Citric acid | 1.0 |
| Phenoxyethanol | 0.7 |
| 1,2 decanediol (symclairol) | 0.5 |
| Safflower oil | 10.0 |

pH adjusted to 5.0 with 10 N NaOH

TABLE 47
Veterinary Composition 3

| Ingredients | Percentage (w/w) |
|---|---|
| Water | 71.04 |
| Xanthum gum | 0.5 |
| Glycerin | 15.0 |
| Bisabolol, ginger root extract | 0.2 |
| (symrelief) | |
| Panthenol (75W) | 0.66 |
| Lemon grass oil | 0.3 |
| Orange oil | 0.1 |
| Lactic acid | 1.0 |
| Phenoxyethanol | 0.7 |
| 1,2 decanediol (symclairol) | 0.5 |
| Fatty acid ester (PCL Liquid 100) | 0.5 |
| Safflower oil | 10.0 |

TABLE 48
Veterinary Composition 4 (teat dip)

| Ingredients | Percentage (w/w) |
|---|---|
| Water | 71.7 |
| Xanthum gum | 0.5 |
| DC Silicone Q2 5220 | 0.5 |
| Glycerin | 15.0 |
| Bisabolol, ginger root extract | 0.2 |
| (symrelief) | |
| Panthenol (75W) | 0.66 |
| Lemon grass oil | 0.3 |
| Orange oil | 0.1 |
| Citric acid | 1.0 |
| Phenoxyethanol | 0.7 |
| 1,2 decanediol (symclairol) | 0.5 |
| Fatty acid ester | 0.5 |
| (PCL Liquid 100) | |
| Safflower oil | 10.0 |

pH adjusted to 5.0 with 10 N NaOH

TABLE 49
Veterinary Composition 5 (teat dip)

| Ingredients | Percentage (w/w) |
|---|---|
| Water | 71.7 |
| Xanthum gum | 0.5 |
| DC Silicone Q2 5220 | 0.5 |
| Glycerin | 15.0 |
| Bisabolol, ginger root extract | 0.2 |
| (symrelief) | |
| Lemon grass oil | 0.3 |
| Orange oil | 0.1 |
| Citric acid | 1.0 |
| Phenoxyethanol | 0.7 |
| 1,2 decanediol (symclairol) | 0.5 |
| Fatty acid ester | 0.5 |
| (PCL Liquid 100) | |
| Safflower oil | 10.0 |

pH adjusted to 5.0 with 10 N NaOH

### 4.11 Household/Industrial Products

In a subset of non-limiting embodiments, the present invention provides for household/industrial products comprising a combination of one or more essential oil and/or IC together with one or more fruit acid, as set forth in section 4.6, 4.7, and 4.8, above.

Non-limiting embodiments of household/industrial products which may utilize the invention include householder cleaners such as concentrated liquid cleaners and spray cleaners, cleaning wipes, dish washing liquid, dish washer detergent, spray-mop liquid, furniture polish, indoor paint, outdoor paint, dusting spray, laundry detergent, fabric softener, rug/fabric cleaner, window and glass cleaner, toilet bowl cleaner, liquid/cream cleanser, etc. In a particular embodiment, the invention may be used in a food wash product, designed to clean fruits and vegetables prior to consumption. “Household products” are products, other than personal care products, that would be used by individual consumers. “Industrial products” refers to products that are used in industry.

Household-industrial compositions according to the invention, in addition to one or more essential oil and/or IC together with one or more fruit acid, may further comprise one or (preferably) more than one component selected from the group consisting of surfactants, builders (e.g., sequestering builders, precipitating builders, ion exchange builders), solvents, thickeners, abrasives, acids, bases (alkalis), antimicrobial agents, soaps, bleaching agents, enzymes, preservatives, and sudsing agents, as well as additional components as may be known in the art.

In various non-limiting embodiments of the invention, a household/industrial product comprising a combination of one or more essential oil and/or IC together with one or more fruit acid may further comprise a surfactant, for example, but not limited to, an anionic surfactant such as an alkyl sulfate, an alkyldiphenyloxide disulfonate salt (e.g., the DOWFAX series by the Dow Chemical Company), an alkylbenzenesulfonate, an alcohol ethoxysulfate; a cationic surfactant; a non-ionic surfactant, such as a secondary alcohol ethoxylate (e.g., the TERGITAOL series by the Dow Chemical Company) or an alkyl polyglucoside (e.g., the TRITON series by the Dow Chemical Company); or an amphoteric surfactant such as an imidazoline or betaine compound.

In various non-limiting embodiments of the invention, a household/industrial product comprising a combination of one or more essential oil and/or IC together with one or more fruit acid may further comprise a builder, for example, but not limited to, a sequestering builder (chelating agent) such as ethylenediaminetetraacetic acid (“EDTA”), sodium citrate, or a complex phosphate; an ion exchange builder such as zeolite, or a precipitating builder such as sodium carbonate or sodium silicate.

In various non-limiting embodiments of the invention, a household/industrial product comprising a combination of one or more essential oil and/or IC together with one or more fruit acid may further comprise a solvent, for example, but not limited to, water, an alcohol such as methanol, ethanol, isopropyl alcohol, or butanol; a hydrocarbon such as an aromatic hydrocarbon, propylene glycol, methylene chloride, acetone, a petroleum distillate, and/or a glycol ether.

In various non-limiting embodiments of the invention, a household/industrial product comprising a combination of one or more essential oil and/or IC together with one or more fruit acid may further comprise a thickener, for example, but not limited to, a polyethylene glycol. a methoxypolyethylene glycol, and/or hydroxyethyl cellulose.

In various non-limiting embodiments of the invention, a household/industrial product comprising a combination of one or more essential oil and/or IC together with one or more fruit acid may further comprise an abrasive, such as, but not limited to, silica, feldspar or calcite.

In various non-limiting embodiments of the invention, a household/industrial product comprising a combination of one or more essential oil and/or IC together with one or more fruit acid may further comprise an acid, such as, but not limited to, acetic acid, hydroacetic acid, phosphoric acid or hydrochloric acid.

In various non-limiting embodiments of the invention, a household/industrial product comprising a combination of one or more essential oil and/or IC together with one or more fruit acid may further comprise a base (alkali) such as, but not limited to, ammonia or sodium bicarbonate.

In various non-limiting embodiments of the invention, a household/industrial product comprising a combination of one or more essential oil and/or IC together with one or more fruit acid may further comprise an antimicrobial agent, for example, but not limited to, compounds as set forth above for personal care compositions, and also pine oil and sodium hypochlorite.

In various non-limiting embodiments of the invention, a household/industrial product comprising a combination of one or more essential oil and/or IC together with one or more fruit acid may further comprise a bleaching agent, for example, but not limited to, sodium hypochlorite, hydrogen peroxide, sodium percarbonate and sodium perborate.

In various non-limiting embodiments of the invention, a household/industrial product comprising a combination of one or more essential oil and/or IC together with one or more fruit acid may further comprise an enzyme, such as, but not limited to, a protease or a lipase.

In various non-limiting embodiments of the invention, a household/industrial product comprising a combination of one or more essential oil and/or IC together with one or more fruit acid may further comprise a preservative, such as, but not limited to, butylated hydroxytoluene, glutaraldehyde, and EDTA.

In various non-limiting embodiments of the invention, a household/industrial product comprising a combination of one or more essential oil and/or IC together with one or more fruit acid may further comprise a sudsing agent, such as, but not limited to, diethanolamine or triethanolamine.

In one set of non-limiting embodiments, the present invention provides for surface cleaner compositions comprising (i) one or more EO/IC, preferably where the EO(s)/IC(s) are selected from the group consisting of lemongrass oil and/or an IC thereof; orange oil and/or an IC thereof; cinnamon leaf oil and/or an IC thereof; basil oil and/or an IC thereof; and/or pine oil and/or an IC thereof; at a total concentration of between about 0.1 and 1 percent (weight/weight); (ii) a fruit acid, preferably citric acid, at a concentration of between about 1 and 2 percent (weight/weight); (iii) an alcohol, preferably ethanol, at a concentration of between about 5 and 20 percent (weight/weight); and (iv) optionally triclosan at a concentration of between about 0.05 and 1 percent (weight/weight), where the ratio of EO(s)/IC(s) to fruit acid is between about 1:1 to 1:10 (inclusive) and the pH is between about 3 and about 7, preferably between 3 and 5. In certain non-limiting embodiments of the invention, cinnamon leaf oil or an IC thereof and/or pine oil or an IC thereof is not present.

In specific, non-limiting embodiments, the present invention provides for the following surface cleaners, having concentrations of active ingredients as indicated, as well as concentrated stock solutions of these formulations which may be diluted to achieve the respective concentrations.

TABLE 50
Surface Cleaners

| Surface Cleaner | Active ingredients |
|---|---|
| Surface Disinfectant-LG cit 2 | 0.2% Lemon grass oil |
| | 2% Citric acid |
| | 7.65% Alcohol |
| | 0.15% surfactants |
| Surface Disinfectant-LG P cit 4 | 0.3% Pine oil |
| | 0.1% Lemon grass oil |
| | 2% Citric Acid |
| | 7.45% alcohol |
| | 0.15% Surfactants |
| Surface Disinfectant-P cit 5 | 0.5% Pine oil |
| | 2% Citric acid |
| | 7.45% alcohol |
| | 0.15% surfactants |
| Surface Disinfectant-PO Cit 7 | 0.5% Pine oil |
| | 0.2% Orange oil |
| | 1% Citric Acid |
| | 5.35% alcohol |
| | 0.15% Surfactants |
| Surface Disinfectant-LGO Cit 7 | 0.5% lemongrass oil |
| | 0.2% Orange oil |
| | 1% Citric Acid |
| | 5.35% alcohol |
| | 0.15% Surfactants |

TABLE 51
Stock solution of hard surface
Disinfectant-LG-O-Cit1 + Dodecanediol:

| Ingredient | Percentage (w/w) |
|---|---|
| Lemon Grass oil | 3.0 |
| Orange oil | 1.0 |
| Citric Acid | 10.0 |
| 1,12 Dodecanediol | 5.0 |
| SDA 40B alcohol | 79.5 |
| Pluronic Surfactant L-61 | 0.5 |
| Pluronic Surfactant F-127 | 0.5 |
| Pluronic Surfactant F-87 | 0.5 |

Before use, this solution is diluted 10 fold with water.

TABLE 52
Stock solution of hard surface Disinfectant-LG-O-Cit
2 + Dodecanediol

| Ingredient | Percentage (w/w) |
|---|---|
| Lemon Grass oil | 3.0 |
| Orange oil | 1.0 |
| Citric Acid | 20.0 |
| 1,12 Dodecanediol | 5.0 |
| SDA 40B alcohol | 69.5 |
| Pluronic Surfactant L-61 | 0.5 |
| Pluronic Surfactant F-127 | 0.5 |
| Pluronic Surfactant F-87 | 0.5 |

Before use, this solution is diluted 10 fold with water.

The detailed description hereby incorporates, by reference, the specific working examples of the invention set forth below.

The working examples sometimes refer to Softsoap® or Dial® soaps.

Softsoap® is a commercially sold liquid soap comprising water, sodium laureth sulfate, cocamidopropyl betaine, decylglucoside, sodium chloride, fragrance, DMDM hydantoin, PEG-120 methyl glucose dioleate, tetrasodium ethylene diamine tetracetic acid, sodium sulfate, polyquaternium-7, citric acid, poloxamer 124, PEG-7 glyceryl, cocoate, benzophenine-4, and colors.

Dial® soap is a commercially sold liquid soap, where Dial® Antibacterial hand soap comprises, as active agent, 0.15 percent triclosan, and the inactive agents are water, sodium laureth sulfate, ammonium lauryl sulfate, decyl glucoside, cocamidopropyl betaine, glycerine, sodium chloride, PEG-18 gylceryl oleate/cocoate, fragrance, cocamide MEA, DMDM hydantoin, tetrasodium ethylene diamine tetracetic acid and colors.

### 4.12 Preservative Compositions

In certain non-limiting embodiments of the invention, the compositions may be formulated as preservative compositions to be used alone or in conjunction with personal care, household or veterinary, products for preservation purposes. Such compositions may contain lemongrass oil, orange oil, lactic or citric acid, phenoxyethanol and/or an alkanediol. Alkanediols include but are not limited to 1,2-decanediol, 1,12-dodecanediol, and/or 1,2-octanediol. The ingredients are combined in an appropriate solvent including but not limited to ethanol, butanol, 3-methoxy-3-methyl-1-butanol, or combinations thereof. The pH of these solutions are adjusted to 5.0, with an appropriate buffer, including for example sodium hydroxide (NaOH). 0.5-5.0% of the preservative compositions can be used in various formulations, preferably 2.0-3.0% of the preservative compositions.

A general formulation for preservative compositions (which may optimally be in the form of stock solutions, which may be diluted prior to use) is as follows. For all preservative compositions, pH is adjusted to 5.0.

TABLE 53
General compositions of preservatives

| | Composition of stock | Composition in products |
|---|---|---|
| Ingredient | solution % (w/w) | % (w/w) |
| Lemongrass oil | 10-25 | 0.3-0.5 |
| Orange oil | 1.6-3.3 | 0.05-0.1 |
| Lactic acid/Citric acid | 3.3-13.4 | 0.1-0.4 |
| Phenoxyethanol | 16.6-33.3 | 0-1.0 |
| Alkanediols | 0-35 | 0-1.0 |
| 3-methoxy-3-methyl- | 0-50 | 0-1.5 |
| 1-butanol | | |
| SDA 40 B alcohol | 0-70 | 0-2.1 |

Specific non-limiting examples of such preservative formulations follow below.

TABLE 54
Preservative composition A

| Ingredient | Percentage (w/w) |
|---|---|
| Lemongrass oil | 10 |
| Farnesol | 10 |
| Orange oil | 5 |
| lactic acid | 7 |
| 1,2 decanediol | 7 |
| SDA 40 B alcohol | 61 |

TABLE 55
Preservative composition B

| Ingredient | Percentage (w/w) |
|---|---|
| Lemongrass oil | 15 |
| Farnesol | 15 |
| Orange oil | 10 |
| lactic acid | 10 |
| SDA 40 B alcohol | 50 |

TABLE 56
Preservative composition C

| Ingredient | Percentage (w/w) |
|---|---|
| Farnesol | 17 |
| Citric acid | 7 |
| 1,2 decanediol | 7 |
| SDA 40 B alcohol | 69 |

TABLE 57
Preservative composition D

| Ingredient | Percentage (w/w) |
|---|---|
| Lemongrass oil | 15 |
| Orange oil | 5 |
| Lactic acid | 10 |
| 1,2 decanediol | 20 |
| 1,2 Octanediol | 20 |
| SDA 40 B alcohol | 30 |

TABLE 58
Preservative composition E

| Ingredient | Percentage (w/w) |
|---|---|
| Lemongrass oil | 15 |
| Orange oil | 5 |
| Lactic acid | 10 |
| 1,2 Octanediol | 40 |
| SDA 40 B alcohol | 30 |

Additional specific non-limiting examples of preservative compositions follow below, with the compositions of stock solutions as well as varying percentages of the preservative compositions in products.

TABLE 59
Preservative composition F

| | Composition of stock | Products containing 2% |
|---|---|---|
| Ingredient | solution % (w/w) | of preservative % (w/w) |
| Lemongrass oil | 25 | 0.5 |
| Orange oil | 5 | 0.1 |
| Lactic acid | 10 | 0.2 |
| Phenoxyethanol | 35 | 0.7 |
| SDA 40 B alcohol | 25 | 0.5 |

TABLE 60
Preservative composition G

| | Composition of stock | Products containing 3% |
|---|---|---|
| Ingredient | solution % (w/w) | of preservative % (w/w) |
| Lemongrass oil | 10 | 0.3 |
| Orange oil | 3.3 | 0.1 |
| Lactic acid | 6.7 | 0.2 |
| Phenoxyethanol | 23.3 | 0.7 |
| 1,2-decanediol | 16.7 | 0.5 |
| SDA 40 B alcohol | 40 | 1.2 |

TABLE 61
Preservative composition H

| | Composition of stock | Products containing 3% |
|---|---|---|
| Ingredient | solution % (w/w) | of preservative % (w/w) |
| Lemongrass oil | 10 | 0.3 |
| Orange oil | 3.3 | 0.1 |
| Lactic acid | 6.7 | 0.2 |
| Phenoxyethanol | 23.3 | 0.7 |
| 1,12-dodecanediol | 16.7 | 0.5 |
| 3-methoxy-3-methyl- | 40 | 1.2 |
| 1-butanol | | |

TABLE 62
Preservative composition I

| | Composition of stock | Products containing 3% |
|---|---|---|
| Ingredient | solution % (w/w) | of preservative % (w/w) |
| Lemongrass oil | 16.7 | 0.5 |
| Orange oil | 3.3 | 0.1 |
| Lactic acid | 6.7 | 0.2 |
| Phenoxyethanol | 23.3 | 0.7 |
| 1,12-dodecanediol | 16.7 | 0.5 |
| 3-methoxy-3-methyl- | 33.3 | 1.0 |
| 1-butanol | | |

TABLE 63
Preservative composition J

| | Composition of stock | Products containing 2.5% |
|---|---|---|
| Ingredient | solution % (w/w) | of preservative % (w/w) |
| Lemongrass oil | 16.7 | 0.5 |
| Orange oil | 3.3 | 0.1 |
| Lactic acid | 6.7 | 0.2 |
| 1,12-dodecanediol | 33.3 | 0.5 |
| 3-methoxy-3-methyl- | 40 | 1.2 |
| 1-butanol | | |

TABLE 64
Preservative composition K

| | Composition of stock | Products containing 2.5% |
|---|---|---|
| Ingredient | solution % (w/w) | of preservative % (w/w) |
| Lemongrass oil | 20 | 0.5 |
| Orange oil | 4 | 0.1 |
| Lactic acid | 8 | 0.2 |
| Octanediol | 40 | 1.0 |
| SDA 40 B | 28 | 0.7 |

TABLE 65
Preservative composition L

| | Composition of stock | Products containing 3% |
|---|---|---|
| Ingredient | solution % (w/w) | of preservative % (w/w) |
| Lemongrass oil | 16.7 | 0.5 |
| Orange oil | 3.3 | 0.1 |
| Lactic acid | 6.7 | 0.2 |
| 1,12-decanediol | 33.3 | 1.0 |
| SDA 40 B | 40.0 | 1.2 |

TABLE 66
Preservative composition M

| | Composition | Products containing |
|---|---|---|
| | of stock | 3% of preservative |
| Ingredient | solution % (w/w) | % (w/w) |
| Lemongrass oil | 10 | 0.3 |
| Orange oil | 3.3 | 0.1 |
| Farnesol | 10 | 0.3 |
| Lactic acid | 6.7 | 0.2 |
| Phenoxyethanol | 20 | 0.6 |
| 1,12-dodecanediol | 16.7 | 0.5 |
| 3-methoxy-3-methyl-1-butanol | 33.3 | 1.0 |

TABLE 67
Preservative composition N

| | Composition of stock | Products containing 2% |
|---|---|---|
| Ingredient | solution % (w/w) | of preservative % (w/w) |
| Lemongrass oil | 25 | 0.5 |
| Orange oil | 5 | 0.1 |
| Citric acid | 25 | 0.5 |
| SDA 40 B | 45 | 0.9 |

TABLE 68
Preservative composition O

| | Composition of stock | Products containing 2.5% |
|---|---|---|
| Ingredient | solution % (w/w) | of preservative % (w/w) |
| Lemongrass oil | 20 | 0.5 |
| Orange oil | 4.0 | 0.1 |
| Citric acid | 20 | 0.5 |
| Phenoxyethanol | 28 | 0.7 |
| SDA 40 B | 28 | 0.7 |

TABLE 69
Preservative composition P

| | Composition of stock | Products containing 2.5% |
|---|---|---|
| Ingredient | solution % (w/w) | of preservative % (w/w) |
| Lemongrass oil | 20 | 0.5 |
| Orange oil | 4.0 | 0.1 |
| Citric acid | 20 | 0.5 |
| 1,2-octanediol | 28 | 0.7 |
| SDA 40 B | 28 | 0.7 |

TABLE 70
Preservative composition Q

| | Composition of stock | Products containing 2.5% |
|---|---|---|
| Ingredient | solution % (w/w) | of preservative % (w/w) |
| Lemongrass oil | 20 | 0.5 |
| Orange oil | 4.0 | 0.1 |
| Citric acid | 20 | 0.5 |
| 1,2-decanediol | 28 | 0.7 |
| SDA 40 B | 28 | 0.7 |

TABLE 71
Preservative composition -1

| | | Products containing 1.75% |
|---|---|---|
| Ingredients | Stock (% w/w) | stock (% w/w) |
| Lemongrass oil | 5.6 | .01 |
| Orange oil | 2.8 | 0.05 |
| Lactic acid | 11.4 | 0.2 |
| Octanediol | 40 | 0.7 |
| Phenoxyethanol | 40 | 0.7 |
| (pH of Stock solution 5.0) | | |

TABLE 72
Preservative composition -2

| | | Products containing 2.0% |
|---|---|---|
| Ingredients | Stock (% w/w) | stock (% w/w) |
| Lemongrass oil | 3.75 | 0.075 |
| Orange oil | 1.25 | 0.025 |
| Lactic acid | 15 | 3.0 |
| Octanediol | 40 | 0.8 |
| Phenoxyethanol | 40 | 0.8 |
| (pH of Stock solution 5.0) | | |

TABLE 73
Preservative composition -3

| | | Products containing 2.0% |
|---|---|---|
| Ingredients | Stock (% w/w) | stock (% w/w) |
| Lemongrass oil | 2.0 | 0.04 |
| Orange oil | 0.5 | 0.01 |
| Lactic acid | 10 | 0.2 |
| Octanediol | 60 | 1.2 |
| Phenoxyethanol | 27.5 | 0.55 |
| (pH of Stock solution 5.0) | | |

TABLE 74
Preservative composition -4

| | | Products containing 2.0% |
|---|---|---|
| Ingredients | Stock (% w/w) | stock (% w/w) |
| Lemongrass oil | 5 | 0.1 |
| Orange oil | 2.5 | 0.05 |
| Lactic acid | 10 | 0.2 |
| Octanediol | 40 | 0.8 |
| Propylene glycol | 42.5 | 0.8 |
| (pH of Stock solution 5.0) | | |

TABLE 75
Preservative composition -5

| | | Products containing 2.0% |
|---|---|---|
| Ingredients | Stock (% w/w) | stock (% w/w) |
| Lemongrass oil | 5 | 0.1 |
| Orange oil | 2.5 | 0.05 |
| Lactic acid | 10 | 0.2 |
| Octanediol | 50 | 1.0 |
| Propylene glycol | 32.5 | 0.65 |
| (pH of Stock solution 5.0) | | |

TABLE 76
Preservative composition -6

| | | Products containing 2.0% |
|---|---|---|
| Ingredients | Stock (% w/w) | stock (% w/w) |
| Lemongrass oil | 3.75 | 0.075 |
| Orange oil | 1.25 | 0.025 |
| Lactic acid | 10 | 0.2 |
| Octanediol | 50 | 1.0 |
| Propylene glycol | 35 | 0.7 |
| (pH of Stock solution 5.0) | | |

TABLE 77
Preservative composition -7

| | | Products containing 2.0% |
|---|---|---|
| Ingredients | Stock (% w/w) | stock (% w/w) |
| Lemongrass oil | 2.5 | 0.05 |
| Orange oil | 1.25 | 0.025 |
| Lactic acid | 10 | 0.2 |
| Octanediol | 50 | 1.0 |
| Propylene glycol | 36.25 | 0.725 |
| (pH of Stock solution 5.0) | | |

TABLE 78
Preservative composition -7-A

| | | Products containing 2.0% |
|---|---|---|
| Ingredients | Stock (% w/w) | stock (% w/w) |
| Lemongrass oil | 2.0 | 0.05 |
| Orange oil | 1.0 | 0.025 |
| Lactic acid | 8.0 | 0.2 |
| Octanediol | 40 | 1.0 |
| Propylene glycol | 49 | 1.225 |
| (pH of Stock solution 5.0) | | |

TABLE 79
Preservative composition -7-B-L

| | | Products containing 2.0% |
|---|---|---|
| Ingredients | Stock (% w/w) | stock (% w/w) |
| Lemongrass oil | 2.8 | 0.07 |
| Lactic acid | 8.0 | 0.2 |
| Octanediol | 40 | 1.0 |
| Propylene glycol | 49.2 | 1.23 |
| (pH of Stock solution 5.0) | | |

TABLE 80
Preservative composition -7-B-M

| | | Products |
|---|---|---|
| | Stock | containing 2.0% |
| Ingredients | (% w/w) | stock (% w/w) |
| Lemongrass oil | 2.8 | 0.07 |
| Multifruit BSC | 8.0 | 0.2 |
| * Mixture of lactic, citric, tartaric, | | |
| glycolic, malic acid extracted from | | |
| plants (obtained from Arch Chemicals) | | |
| Octanediol | 40 | 1.0 |
| Propylene glycol | 49.2 | 1.23 |
| (pH of Stock solution 5.0) | | |

The following Tables provide the formulations of specific preservative compositions containing grapefruit seed extract and grape seed extract.

TABLE 81
Preservative composition -G-8

| | | Products containing 2.0% |
|---|---|---|
| Ingredients | Stock (% w/w) | stock (% w/w) |
| Grapefruit seed extract | 10 | 0.2 |
| Lactic acid | 10 | 0.2 |
| Octanediol | 50 | 1.0 |
| Propylene glycol | 30 | 0.6 |
| (pH of Stock solution 5.0) | | |

TABLE 82
Preservative composition -G-9

| | | Products containing 2.0% |
|---|---|---|
| Ingredients | Stock % w/w) | stock (% w/w) |
| Grapefruit seed extract | 10 | 0.2 |
| Lactic acid | 10 | 0.2 |
| Octanediol | 50 | 1.0 |
| Safflower oil | 30 | 0.6 |
| (pH of Stock solution 5.0) | | |

TABLE 83
Preservative composition -G-10

| | | Products containing 2.0% |
|---|---|---|
| Ingredients | Stock (% w/w) | stock (% w/w) |
| Lemongrass oil | 2.5 | 0.05 |
| Orange oil | 1.25 | 0.025 |
| Grape seed Extract | 10 | 0.2 |
| Lactic acid | 10 | 0.2 |
| Octanediol | 50 | 1.0 |
| Propylene glycol | 26.25 | 0.525 |
| (pH of Stock solution 5.0) | | |

TABLE 84
Preservative composition -G-10-A

| | | Products containing 2.0% |
|---|---|---|
| Ingredients | Stock (% w/w) | stock (% w/w) |
| Lemongrass oil | 2.0 | 0.05 |
| Grape seed Extract | 8.0 | 0.2 |
| Lactic acid | 8.0 | 0.2 |
| Octanediol | 40 | 1.0 |
| Propylene glycol | 42.0 | 1.05 |
| (pH of Stock solution 5.0) | | |

TABLE 85
Preservative composition -G-10-M

| | | Products containing |
|---|---|---|
| Ingredients | Stock (% w/w) | 2.0% stock (% w/w) |
| Lemongrass oil | 2.5 | 0.05 |
| Orange Oil | 1.25 | 0.025 |
| Grape seed Extract | 10 | 0.2 |
| Multifruit BSC | 10 | 0.2 |
| *Mixture of lactic, citric, tartaric, | | |
| glycolic, malic acid extracted from | | |
| plants(obtained from Arch | | |
| Chemicals) | | |
| Octanediol | 50 | 1.0 |
| Propylene glycol | 26.25 | 0.525 |
| (pH of Stock solution 5.0) | | |

TABLE 86
Preservative composition -G-10-C

| | | Products containing 2.0% |
|---|---|---|
| Ingredients | Stock (% w/w) | stock (% w/w) |
| Lemongrass oil | 2.5 | 0.05 |
| Orange Oil | 1.25 | 0.025 |
| Grape seed Extract | 10 | 0.2 |
| Citric acid | 10 | 0.2 |
| Octanediol | 50 | 1.0 |
| Propylene glycol | 26.25 | 0.525 |
| (pH of Stock solution 5.0) | | |

TABLE 87
Preservative composition -G-11

| | | Products containing 2.0% |
|---|---|---|
| Ingredients | Stock (% w/w) | stock (% w/w) |
| Lemongrass oil | 3.75 | 0.075 |
| Orange Oil | 1.25 | 0.025 |
| Grape seed Extract | 10 | 0.2 |
| Lactic Acid | 10 | 0.2 |
| Octanediol | 50 | 1.0 |
| Propylene glycol | 25 | 0.5 |
| (pH of Stock solution 5.0) | | |

TABLE 88
Preservative composition -G-12

| | | Products containing 2.0% |
|---|---|---|
| Ingredients | Stock (% w/w) | stock (% w/w) |
| Grape seed Extract | 15 | 0.3 |
| Lactic acid | 10 | 0.2 |
| Octanediol | 50 | 1.0 |
| Propylene glycol | 25 | 0.5 |
| (pH of Stock solution 5.0) | | |

TABLE 89
Preservative composition -G-13

| | | Products containing 2.0% |
|---|---|---|
| Ingredients | Stock (% w/w) | stock (% w/w) |
| Lemongrass oil | 12 | 0.3 |
| Orange Oil | 4 | 0.1 |
| Grape seed Extract | 8.0 | 0.2 |
| Lactic Acid | 8.0 | 0.2 |
| Octanediol | 40 | 1.0 |
| Propylene glycol | 28 | 0.7 |
| (pH of Stock solution 5.0) | | |

TABLE 90
Preservative composition -G-14

| | | Products containing 2.0% |
|---|---|---|
| Ingredients | Stock (% w/w) | stock (% w/w) |
| Lemongrass oil | 0.8 | 0.02 |
| Orange Oil | 0.4 | 0.01 |
| Grape seed Extract | 12 | 0.3 |
| Lactic Acid | 8.0 | 0.2 |
| Octanediol | 40 | 1.0 |
| Propylene glycol | 38.8 | 0.97 |
| (pH of Stock solution 5.0) | | |

TABLE 91
Preservative composition -G-15

| | | Products containing 2.0% |
|---|---|---|
| Ingredients | Stock (% w/w) | stock (% w/w) |
| Lemongrass oil | 0.8 | 0.075 |
| Grape seed Extract | 12 | 0.2 |
| Lactic acid | 8.0 | 0.2 |
| Octanediol | 40 | 1.0 |
| Propylene glycol | 41.2 | 1.03 |
| (pH of Stock solution 5.0) | | |

TABLE 92
Preservative composition -G-16

| | | Products containing 2.5% |
|---|---|---|
| Ingredients | Stock (% w/w) | stock (% w/w) |
| Lemongrass oil | 2.8 | 0.07 |
| Grape seed Extract | 8.0 | 0.2 |
| Lactic Acid | 8.0 | 0.2 |
| Octanediol | 28 | 0.7 |
| Propylene glycol | 53.2 | 1.33 |
| (pH of Stock solution 5.0) | | |

TABLE 93
Preservative composition -G-17

| | | Products containing 2.5% |
|---|---|---|
| Ingredients | Stock (% w/w) | stock (% w/w) |
| Lemongrass oil | 2.8 | 0.07 |
| Grape seed Extract | 8 | 0.2 |
| Lactic Acid | 8 | 0.2 |
| Octanediol | 40 | 1.0 |
| Propylene glycol | 41.2 | 1.03 |
| (pH of Stock solution 5.0) | | |

The following Tables provide the formulations of specific preservative compositions containing essential oils/botanical extracts, fruit acids and alkanediol without solvents. All of the compositions ending in “L” are noted as the L series, which contain lactic acid. All of the compositions ending in “M” are noted as the M series, which contain Multifruit®BSC. Multifruit®BSC contains a mixture of lactic, citric, tartaric, glycolic, and malic acid extracted from plants (obtained from Arch Chemicals). The pH of all the preservative compositions in the following Tables were adjusted to 5.0 with 10 N. NaoH (30-70 ul/ml were required).

TABLE 94
Preservative composition -6-L

| Ingredients | Stock (% w/w) | Cream containing 1.3% stock |
|---|---|---|
| Lemongrass oil | 5.78 | 0.075 |
| Orange oil | 1.92 | 0.025 |
| Lactic Acid | 15.4 | 0.2 |
| Octanediol | 76.9 | 1.0 |

TABLE 95
Preservative composition -6-M

| Ingredients | Stock (% w/w) | Cream containing 1.3% stock |
|---|---|---|
| Lemongrass oil | 5.78 | 0.075 |
| Orange oil | 1.92 | 0.025 |
| Multifruit extract | 15.4 | 0.2 |
| Octanediol | 76.9 | 1.0 |

TABLE 96
Preservative composition-10-G-L

| Ingredients | Stock (% w/w) | Cream containing 1.5% stock |
|---|---|---|
| Lemongrass oil | 3.3 | 0.05 |
| Orange oil | 1.6 | 0.025 |
| Grapefruit seed extract | 15.0 | 0.225 |
| Lactic Acid | 13.3 | 0.2 |
| Octanediol | 66.8 | 1.0 |

TABLE 97
Preservative composition-10-G-M

| | | Cream containing |
|---|---|---|
| Ingredients | Stock (% w/w) | 1.5% stock |
| Lemongrass oil | 3.3 | 0.05 |
| Orange oil | 1.6 | 0.025 |
| Grapefruit seed extract | 15 | 0.225 |
| Multifruit BSC | 13.3 | 0.2 |
| Mixture of lactic, citric, tartaric, | | |
| glycolic, malic acid extracted from | | |
| plants (obtained from Arch | | |
| Chemicals) | | |
| Octanediol | 66.8 | 1.0 |

TABLE 98
Preservative composition-11-G-L

| Ingredients | Stock (% w/w) | Cream containing 1.5% stock |
|---|---|---|
| Lemongrass oil | 5.0 | 0.075 |
| Orange oil | 1.7 | 0.025 |
| Grapefruit seed extract | 13.3 | 0.2 |
| Lactic Acid | 13.3 | 0.2 |
| Octanediol | 66.7 | 1.0 |

TABLE 99
Preservative composition-11-G-M

| | | Cream containing |
|---|---|---|
| Ingredients | Stock (% w/w) | 1.5% stock |
| Lemongrass oil | 5.0 | 0.075 |
| Orange oil | 1.7 | 0.025 |
| Grapefruit seed extract | 13.3 | 0.2 |
| Multifruit BSC | 13.3 | 0.2 |
| Mixture of lactic, citric, tartaric, | | |
| glycolic, malic acid extracted from | | |
| plants (obtained from Arch | | |
| Chemicals) | | |
| Octanediol | 66.7 | 1.0 |

TABLE 100
Preservative composition-G-17-G

| Ingredients | Stock (% w/w) | Cream containing 2.5% stock |
|---|---|---|
| Lemongrass oil | 2.8 | 0.07 |
| Grapefruit seed extract | 8.0 | 0.2 |
| Lactic acid | 8.0 | 0.2 |
| Octanediol | 40 | 1.0 |
| Glycerine | 41.2 | 1.03 |

TABLE 101
Preservative composition-G-18-G

| | Composition of Stock | Cream containing |
|---|---|---|
| Ingredients | Solution (% w/w) | 2.5% stock |
| Lemongrass oil | 4.67 | 0.07 |
| Grapefruit seed extract | 13.33 | 0.2 |
| Lactic acid | 13.33 | 0.2 |
| Octanediol | 46.67 | 0.7 |
| Glycerine | 22.0 | 0.33 |
| pH of stock solution is 5.0 | | |

TABLE 102
Preservative composition-G-18

| | Composition of Stock | Cream containing |
|---|---|---|
| Ingredients | Solution (% w/w) | 2.5% stock |
| Lemongrass oil | 4.67 | 0.07 |
| Grapefruit seed extract | 13.33 | 0.2 |
| Lactic acid | 13.33 | 0.2 |
| Octanediol | 46.67 | 0.7 |
| Propylene glycol | 22.0 | 0.33 |

TABLE 103
Preservative composition-G-19-G

| | Composition of Stock | Cream containing |
|---|---|---|
| Ingredients | Solution (% w/w) | 2.5% stock |
| Lemongrass oil | 4.67 | 0.07 |
| Grapefruit seed extract | 13.33 | 0.2 |
| Lactic acid | 13.33 | 0.2 |
| Octanediol | 46.67 | 0.5 |
| Glycerine | 22.0 | 0.53 |

TABLE 104
Preservative composition-G-19

| | Composition of Stock | Cream containing |
|---|---|---|
| Ingredients | Solution (% w/w) | 2.5% stock |
| Lemongrass oil | 4.67 | 0.07 |
| Grapefruit seed extract | 13.33 | 0.2 |
| Lactic acid | 13.33 | 0.2 |
| Octanediol | 46.67 | 0.5 |
| Propylene glycol | 22.0 | 0.53 |
| pH of stock solution is 5.0 | | |

### 5. EXAMPLES

### Example 1

Various concentrations of basil oil and acetic, lactic, and citric acids, separately and in combination, were prepared in 10 percent SDA40-B alcohol and water, and adjusted to 100 percent. Except for citric acid, which was added by weight, all other ingredients were measured by volume. 0.9 ml of each solution were dispensed in sterile culture tubes, in triplicate, and 0.1 ml of a 10⁷ cfu/ml S. aureus culture was added to the tubes, vortexed, and then, five minutes later, 9.0 ml of drug inactivating medium was added to each tube. Serial dilutions were made with the drug inactivating medium. 0.5 ml of the dilutions were plated on trypticase soy agar (“TSA”) plates. As a control, water containing 10 percent SDA40-B alcohol was processed in parallel. The plates were incubated at 37° C. for 24-48 hours and then the colony counts were determined. The results are shown in Table 105. The greater synergy was observed between basil oil and citric acid (“CA”).

TABLE 105

| Compounds | Log 10 Reduction* |
|---|---|
| 1% Basil oil | 2.7 |
| 0.5% Basil oil | 1.8 |
| 0.25% Basil oil | 0.4 |
| 1% Acetic acid (AA) | 0.2 |
| 1% Lactic acid (LA) | 2.3 |
| 1% Citric Acid (CA) | 0.1 |
| 1% Basil oil + 1% LA | 5.4 |
| 1% Basil oil + 1% AA | 3.4 |
| 1% Basil oil + 1% CA | 5.1 |
| 0.5% Basil oil + 1% CA | 5.1 |
| 0.5% Basil oil + 0.5% CA | 5.0 |
| 0.25% Basil oil + 1% CA | 5.0 |
| 0.25% Basil oil + 0.5% CA | 2.5 |

*Log reduction from control bacterial counts ranging from 1 × 10⁶ to 5 × 10⁶.

The same methodology was used to test the antimicrobial activity of combinations of citric acid with other essential oils. The results are shown in Table 106. In these experiments, cinnamon oil and citronella oil exhibited superior antimicrobial activities in combination with citric acid.

TABLE 106

| Compounds | Log 10 Reduction* |
|---|---|
| 1% CA | 0.1 |
| 0.5% Cinnamon bark | 2.4 |
| 0.25% Cinnamon bark | 1.1 |
| 0.5% Cinnamon bark + 1% CA | 5.9 |
| 0.25% Cinnamon bark + 1% CA | 4.3 |
| 0.125% Cinnamon bark + 1% CA | 2.6 |
| 0.25% Cinnamon leaf | 2.8 |
| 0.25% Cinnamon leaf + 1% CA | 5.7 |
| 0.125% Citronella oil | 1.3 |
| 0.25% Citronella oil + 1% CA | 6.2 |
| 0.125% Citronella oil + 1% CA | 3.3 |
| 0.25% Orange oil | 0 |
| 0.25% Orange oil + 1% CA | 2.3 |
| 0.25% Lemon oil | 0.05 |
| 0.25% Lemon oil + 1% CA | 3.3 |
| 0.25% Lavender oil | 0.25 |
| 0.25% Lavender oil + 1% CA | 4.0 |
| 0.25% Clove oil | 0.1 |
| 0.25% Clove oil + 1% CA | 3.3 |
| 0.25% Tea tree oil | 0 |
| 0.25% Tea tree + 1% CA | 4.7 |
| 0.25% Farnesol | 0 |
| 0.25% Farnesol + 1% CA | 4.0 |

*Log reduction from control bacterial counts ranging from 1 × 10⁶ to 5 × 10⁶.

Next, the same general protocol was used to test the efficacy of basil, cinnamon and citronella oils against a variety of organisms, namely E. coli, P. aeruginosa, MRSA, C. albicans, and S. aureus. The results, which demonstrates that in these experiments, combinations of cinnamon oil and citric acid exhibited superior antimicrobial action, are shown in Table 107.

TABLE 107
Log 10 Reductions*

| Compounds | E. coli | P. aeruginosa | MRSA | C. albicans | S. aureus |
|---|---|---|---|---|---|
| 0.25% Basil oil + 1% CA | 6.0 | 5.5 | 5.2 | 1.5 | 5.0 |
| 0.25% Cinnamon bark oil + 1% CA | 6.0 | 6.0 | 5.2 | 4.5 | 4.3 |
| 0.25% Citronella oil + 1% CA | 6.4 | 6.4 | 6.5 | 1.0 | 6.3 |
| 0.25% Cinnamon leaf oil + 1% CA | 6.4 | 6.4 | 6.5 | 5.4 | 6.3 |
| 0.25% Eugenol + 1% CA | — | 6.5 | — | 5.5 | — |

*Log reduction from control bacteria counts ranging from 1 × 10⁶ to 5 × 10⁶ or C. albicans ranging from 1 × 10⁵ to 5 × 10⁵.

### Example 2

The following experiments were performed to evaluate the effectiveness of a hard surface cleaner composition comprising cinnamon leaf oil and citric acid.

Two stock solutions of a hard surface cleaner/disinfectant was prepared, with the following ingredients (the two solutions contained different amounts of cinnamon leaf oil, and therefore the amount of alcohol to bring the solution to 100% also varied).

TABLE 108

| Ingredient | Percentage (w/w) |
|---|---|
| Cinnamon leaf oil | 3.6 or 7.2% |
| Citric acid | 14.3% |
| SDA 40B alcohol | 77.2 or 75.49% |
| | (to bring the volume to 100%) |
| Pluronic surfactant L-61 | 0.7% |
| Pluronic surfactant F-127 | 0.7% |
| Pluronic surfactant F-87 | 0.7% |
| Orange oil | 2.8% |

7% of the stock hard disinfectant was diluted with water to 100%.

0.1 ml of culture containing approximately 1×10⁷ colony forming units (“cfu”) per milliliter was spread evenly on the surface of 2.5×11 cm² tiles using a glass rod and left at room temperature for 10 minutes to dry. After 10 minutes 0.3 ml of the diluted surface disinfectant was spread evenly on the tiles with a sterile glass rod and left for another 10 minutes to dry. The tiles were rinsed with 9.6 ml of inactivating medium (BPBNS), which was collected for testing. The collected medium was serially diluted and 0.5 ml was plated onto TSA plates and incubated at 37° C. for 18-24 hours. The colonies on the plates were counted and the values converted to log₁₀. Commercially available Pinesol®, which contains pine oil, was used as a basis for comparison. Pinesol® containing 15% pine oil was diluted with water as per the manufacturer's instructions to a final concentration of 0.9% pine oil. The results are shown in Table 109. The results show that the composition comprising 0.5% cinnamon leaf oil and 1% citric acid exhibited greater antimicrobial activity than the pine oil cleaner against 4 out of 5 microbes tested.

TABLE 109
Log 10 Reductions*

| | 0.25% cinn. oil + | 0.5% cinn. oil + | |
|---|---|---|---|
| Organism | 1% CA | 1% CA | 0.1% Pinesol ® |
| E. coli | 5.3 | 5.3 | 5.7 |
| P. aeruginosa | 6.1 | 6.1 | 3.9 |
| MRSA | 2.3 | 3.4 | 2.2 |
| C. albicans | 2.5 | 5.2 | 2.1 |
| S. aureus | 3.7 | 4.1 | 2.4 |

*log₁₀ reduction from control bacterial counts (ranges from 1 × 10⁶-5 × 10⁶ for all bacteria, but for C. albicans counts were 1 × 10⁵-5 × 10⁵.

### Example 3

Various concentrations of cinnamon leaf oil and citric acid were dissolved in SDA 40-B alcohol (10%) and water, and adjusted to 100 percent. Except for citric acid, which was added by weight, all other ingredients were measured by volume. 0.9 ml of each solution were dispensed in sterile culture tubes, in triplicate, and 0.1 ml of 10⁷ cfu/ml of S. aureus culture was added to the tubes, vortexed, and then, five minutes later, 9.0 ml of drug inactivating medium was added to each tube. Serial dilutions were made with the drug inactivating medium. 0.5 ml of the dilutions were plated on trypticase soy agar (“TSA”) plates. As a control, water containing 10% percent SDA40-B alcohol was processed in parallel. The plates were incubated at 37° C. for 24-48 hours and then the colony counts were determined. The results are shown in Table 110.

TABLE 110

| Compounds | Log 10 reduction |
|---|---|
| Citric Acid 2% | 0.32 |
| Citric Acid 1.0% | 0.30 |
| Citric Acid 0.5% | 0.20 |
| Citric Acid 0.25% | 0.08 |
| Citric Acid 0.125% | 0.02 |
| Cinnamon leaf oil 0.25% | 0.52 |
| Cinnamon leaf oil 0.5% | 0.55 |
| 0.25% Cinnamon + 0.25% CA | 0.73 |
| 0.25% Cinnamon + 0.5% CA | 3.0 |
| 0.25% Cinnamon + 1.0% CA | 5.6 |
| 0.5% Cinnamon + 0.125% CA | 0.84 |
| 0.5% Cinnamon + 0.25% CA | 2.2 |
| 0.5% Cinnamon + 0.5% CA | 3.2 |
| 0.5% Cinnamon + 1.0% CA | 6.5 |
| 0.5% Cinnamon + 2.0% CA | 6.7 |

### Example 4

A liquid soap, called “CN1-A” containing cinnamon oil and citric acid was prepared, having the following composition.

TABLE 111

| Ingredient | Percentage (w/w) |
|---|---|
| Deionized water | 59.15% |
| Polyox N 60K | 0.2% |
| Pluronic F 87 Prill | 2.0% |
| Ucare Jr 30 | 0.4% |
| D,L Panthenol 50 W | 1.0% |
| Incromide oxide L | 3.0% |
| Crosultane C-50 | 3.0% |
| Montalene C 40 | 3.0% |
| 2-Phenoxy-ethanol | 1.0% |
| Glycerine | 2.0% |
| SDA-40B alcohol | 15.5% |
| Cinnamon leaf oil | 0.5% |
| Citric acid | 1.0% |
| Orange oil | 0.2% |
| Distilled water | 7.95% |

To prepare the soap, cinnamon oil orange oil, citric acid, and phenoxyethanol are dissolved in the alcohol, the remaining ingredients are dissolved in/mixed with water, and then the alcohol and water solutions are mixed. The pH of the mixture was then adjusted to between 5.5 and 6.5 with 0.1 N NaOH.

The antimicrobial activity of the above soap was tested in parallel with commercial Softsoap® containing triclosan (Softsoap® Antibacterial; Colgate-Palmolive). 0.1 ml of a 10⁸ cfu/ml culture of each microbe tested was mixed with 0.1 ml of bovine serum and placed in a sterile culture tube. 0.8 ml of the test soap formulation was added to the tube and vortexed for 30 seconds. Then 9.0 ml DNB was added to neutralize the activity of the soap. The tube was then vortexed and serially diluted with DNB. 0.5 ml of the diluted solution was plated on TSA plates. The same soap base lacking cinnamon oil, citric acid, and orange oil, with phosphate buffered saline mixed with the culture, were used as the controls. The results are shown in Table 112.

TABLE 112

Log₁₀ reduction from control*

| Organisms | CN-1A | Softsoap ®(0.15% TC) |
|---|---|---|
| S. aureus | 2.0 | 0.33 |
| P. aeruginosa | 2.5 | 0.6 |
| E. coli | 4.86 | 0.5 |
| MRSA | 2.7 | 0.8 |
| C. albicans | 1.43 | 0.0 |

*Log₁₀ reduction from control microbe counts which in all cases ranged from 1 × 10⁷-5 × 10⁷.

### Example 5

A liquid soap, called “CN1-B” containing cinnamon oil and citric acid was prepared, having the following composition.

TABLE 113

| Ingredient | Percentage (w/w) |
|---|---|
| Deionized water | 63.2% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerine | 2.0% |
| SDA-40B alcohol | 15.5% |
| Cinnamon leaf oil | 0.5% |
| Citric acid | 1.0% |
| Orange oil | 0.2% |
| Distilled water | 7.8% |

To prepare the soap, cinnamon oil orange oil, citric acid, and phenoxyethanol are dissolved in the alcohol, the remaining ingredients are dissolved in/mixed with water, and then the alcohol and water solutions are mixed. The pH of the mixture was then adjusted to between 5.5 and 6.5 with 0.1 N NaOH.

The antimicrobial activity of the above soap was tested in parallel with commercial Dial® Antibacterial Hand Soap) containing triclosan. 0.1 ml of a 10⁸ cfu/ml culture of each microbe tested was mixed with 0.1 ml of bovine serum and placed in a sterile culture tube. 0.8 ml of the test soap formulation was added to the tube and vortexed for 30 seconds. Then 9.0 ml DNB was added to neutralize the activity of the soap. The tube was then vortexed and serially diluted with DNB. 0.5 ml of the diluted solution was plated on TSA plates. The same soap base lacking cinnamon oil, citric acid, and orange oil, with phosphate buffered saline mixed with the culture, were used as the controls. The results are shown in Table 114.

TABLE 114

Log₁₀ reduction from control*

| Organisms | CN1-B | Dial ® soap (0.15% TC) |
|---|---|---|
| S. aureus | 5.0 | 0.36 |
| MRSA | 5.1 | 0.03 |
| E. coli | 4.45 | 0 |
| P aeruginosa | 5.9 | 0.12 |

*Log₁₀ reduction from control microbe counts which in all cases ranged from 1 × 10⁷ to 5 × 10⁷ 3.4 × 10⁶ for S. aureus, 3-5 × 10⁶ for E. coli and 6 × 10⁵-1.3 × 10⁶ for MRSA.

### Example 6

The effectiveness of Softsoap® Juicy Melon (Colgate-Palmolive) with added cinnamon oil, citric acid, and/or triclosan, against MRSA was evaluated. Testing was performed essentially as set forth in the preceding example. The results are shown in Table 115.

TABLE 115

| Compounds | Log 10 reduction* |
|---|---|
| Soft Soap ® + 1.5% cin-cit | 3.63 |
| Soft soap ® + 0.075% TC | 0.15 |
| Soft soap ® + 0.15% TC | 0.20 |
| Soft soap ® + 0.3% TC | 0.58 |
| Soft soap ® + 0.075% TC + 1.5% Cin-Cit | 4.29 |
| Soft soap ® + 0.15% TC + 1.5% Cin-Cit | 4.87 |
| Soft soap ® + 0.3% TC + 1.5% Cin-Cit | 6.38 |

*Log₁₀ reduction from control microbe counts which in all cases ranged from 1 × 10⁶-5 × 10⁶.

### Example 7

The ability of cinnamon oil and citric acid to potentiate the activity of commercial triclosan-containing soaps such as Softsoap® and Dial® Antibacterial Hand Soap containing 0.15% triclosan was tested using an assay essentially as set forth in Example 5, above. The results are shown in Table 116.

TABLE 116

| | Log₁₀ reduction from control* | | |
|---|---|---|---|
| | S. aureus | E. Coli | MRSA |
| Soft Soap ®-TC | 0.33 | 0.25 | 0.37 |
| Soft Soap ®-TC + CIN- | 3.9 | 3.93 | 6.0 |
| Cit | | | |
| Dial ® Soap-TC | 0.36 | 0 | 0.24 |
| Dial ® Soap-TC + Cin-Cit | 3.74 | 4.18 | 6.0 |

*Log reduction from control bacterial counts (ranges from 3 4 × 10⁶ for S. aureus, 3-5 × 10⁶ for E. coli and 6 × 10⁵-1.3 × 10⁶ for MRSA.

In these experiments, the combination of cinnamon oil and citric acid was found to substantially improve the antimicrobial activity of the commercial soap.

### Example 8

Because a major ingredient of cinnamon oil is eugenol, the effect of adding eugenol on the antimicrobial activity of commercial soaps was also tested. The assay was essentially as set forth in Example 5, above. The results are shown in Table 117.

TABLE 117

| | Log reduction from control* |
|---|---|
| | S. aureus |
| Dial ® Soap-TC | 0.30 |
| Dial ® Soap-TC + 0.5% | 2.32 |
| Eugenol + 1% CA | |
| Dial ® Soap-TC + 0.0.5% | 3.94 |
| cinnamon oil + 1% CA | |

*Log reduction from control bacterial counts (ranged from 3-4 × 10⁶ for S. aureus).

These experiments showed that while adding eugenol improved the antimicrobial effect, the improvement was not as great as that observed for cinnamon oil.

### Example 9

The following experiments were performed to evaluate the antibacterial activity of LG and Citric acid dissolved in alcohol, where the test organism used was S. aureus. Various amounts of LG oil and Citric acid were dissolved in SDA40-B alcohol, and then water was added to result in the EO concentration shown and an alcohol concentration of 10 percent. 0.9 ml of each solution were dispensed in sterile culture tubes, in triplicate, and 0.1 ml of a 10⁷ cfu/ml S. aureus culture was added to the tubes, vortexed, and then, five minutes later, 9.0 ml of drug inactivating medium was added to each tube. Serial dilutions were made with drug inactivating medium. 0.5 ml of the dilutions were plated on trypticase soy agar (“TSA”) plates. As a control, water containing 10 percent SDA40-B alcohol was processed in parallel. The plates were incubated at 37° C. for 24-48 hours and then the colony counts were determined. The results are shown in Table 118.

TABLE 118

| Compounds | Log 10 reduction from control |
|---|---|
| 1% Citric acid | 0.3 |
| 0.5% LG oil | 1.24 |
| 0.55 LG oil + 1% Citric acid | 5.59 |

*Log 10 reduction from control bacterial counts (control counts ranges from 1 × 10⁶ to 5 × 10⁶)

The results shown in Table 118 indicate that LG oil exhibits superior anti bacterial action in combination with Citric acid.

### Example 10

Soaps were prepared containing one or more essential oil, 1% citric acid, and a soap base containing surfactants, emollients, thickeners etc. The pH of the Soaps ranged from 3.2-3.3.

TABLE 119
Soap Containing Lemongrass oil, and Citric acid (LG-Cit-4)
(4 represents total oil 0.4%)

| Ingredient | Percentage (w/w) |
|---|---|
| Deionized water | 63.5% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Lemongrass oil | 0.4% |

TABLE 120
Soap Containing Lemongrass oil, and Citric acid (LG-Cit-6)
(6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Lemongrass oil | 0.6% |

TABLE 121
Soap Containing Lemongrass oil, Orange oil (O oil)
and Citric acid (LGO-Cit 6)
(6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Lemongrass oil | 0.4% |
| Citric acid | 1.0% |
| Orange oil | 0.2% |

TABLE 122
Soap Containing Lemon grass oil , Orange oil
and Citric acid (LGO-Cit 7)
(7 represents total oil 0.7%)

| Ingredient | Percentage (w/w) |
|---|---|
| Deionized water | 63.2% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Lemongrass oil | 0.5% |
| Citric acid | 1.0% |
| Orange oil | 0.2% |

TABLE 123
Soap Containing Cinnamon oil, Orange
oil and Citric acid ( CO-Cit 6)
(6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Cinnamon oil | 0.4% |
| Citric acid | 1.0% |
| Orange oil | 0.2% |

TABLE 124
Soap Containing Cinnamon oil, Orange oil and Citric acid ( CO-Cit 7)
(7 represent total oil 0.7%)

| Ingredient | Percentage (w/w) |
|---|---|
| Deionized water | 63.2% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Cinnamon oil | 0.5% |
| Citric acid | 1.0% |
| Orange oil | 0.2% |

TABLE 125
Soap Containing Orange oil and Citric acid (O-Cit 2)
(2 represents total oil 0.2%)

| Ingredient | Percentage (w/w) |
|---|---|
| Deionized water | 63.7% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Citric acid | 1.0% |
| Orange oil | 0.2% |

TABLE 126
Soap Containing Basil oil (“B oil”), Orange
oil (“O oil”) and Citric acid ( BO-Cit 6)
(6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Basil oil | 0.4% |
| Citric acid | 1.0% |
| Orange oil | 0.2% |

TABLE 127
Soap Containing Citronella oil (“CR oil”), Orange oil(“O oil”), and
Citric acid (CRO-Cit 6)
(6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Citronella oil | 0.4% |
| Citric acid | 1.0% |
| Orange oil | 0.2% |

### Example 11

Certain soaps prepared in Example 14 were tested for antimicrobial activity.

The following method was used. A mixture of 0.1 ml of 10⁷ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hours and the colony counts were determined. Soft Soap® and Dial® soaps containing 0.15% triclosan was also tested similarly at the same time. The soap base without essential oils and Citric acid containing the culture were used as controls. The results, showing 30 second kill activity, are shown in Table 128.

TABLE 128

| Soap | Log 10 reduction from control* |
|---|---|
| LG-Cit 4 | 3.9 |
| LG-Cit 6 | 4.2 |
| O-Cit 2 | 1.5 |
| LGO Cit 6 | 6.4 |

*Log 10 reduction from control bacterial counts (control counts ranges from 1 × 10⁶ to 5 × 10⁶)

These data show that when citric acid was used in combination with 0.4% LG oil+0.2% O oil (LGO-Cit 6) superior antibacterial activity was observed as compared to that of combination of Citric acid and LG oil 0.6% (LG-Cit 6) or the additive activity of Citric acid+0.4% LG oil (LG-Cit 4) and Citric acid+0.2% Orange oil (O-Cit 2).

### Example 12

Certain soaps described in Example 10 were tested for antimicrobial activity.

The following method was used. A mixture of 0.1 ml of 10⁷ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. Soft Soap® and Dial® soaps containing 0.15% triclosan was also tested similarly at the same time. The soap base without essential oils and citric acid containing the culture were used as controls. The results, showing 30 second kill activity, are shown in Table 129.

TABLE 129

| Soap | Log 10 reduction from control* |
|---|---|
| LGO-Cit 6 | 6.4 |
| LGO-Cit 7 | 6.5 |
| CO-Cit 6 | 5.1 |
| CO-Cit 7 | 5.2 |
| BO-Cit 6 | 2.87 |
| CRO-Cit 6 | 4.57 |

*Log 10 reduction from control bacterial counts (ranges from 1 × 10⁶ to 3 × 10⁶.)

These data show that LGO-Cit Soaps were found to exhibit higher antibacterial activity compared to the other essential oil/citric acid combination soaps tested.

### Example 13

The following experiments were performed to evaluate the antibacterial activity of triclosan, LG oil, and combinations of triclosan and LG oil.

Patent application WO/2007/077573 by Mukhopadhyay et al. describes an antimicrobial composition containing triclosan and an essential oil where the ratio of triclosan to the essential oil is 1:5 to 1:100 and the preferred ratio range is 1:10 to 1:90. In the example provided in United States Patent Application Publication No. 20050019431 by Modak et al., triclosan and essential oil at 1:1 ratio showed neither synergistic nor enhanced activity.

Triclosan (“TC”) is often used in personal care products at a concentration of 0.15-0.3%. In order to determine whether or not TC at this concentration would enhance the activity of essential oil at 0.4-0.7% which is the concentration used in various formulations described in this application, the antibacterial activity of soaps containing triclosan; LG oil; or TC and LG oil at TC:LG weight ratios of 1:1.7 to 1:4.6 were evaluated.

To prepare the soaps, TC, LG oil or their combination were dissolved in SDA40 B alcohol and then added to Softsoap® (a formulation lacking triclosan), then diluted with water, where the amount of SDA40B alcohol used represented 5.5% of the final solution and the amount of Softsoap® used represented 92% of the final solution. Soft Soap® was used as the control in this study.

The following method was used. A mixture of 0.1 ml of 10⁸ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hours and the colony counts were determined. The results are shown in Table 130.

TABLE 130

| Soap | Log 10 reduction from control* |
|---|---|
| Softsoap ® + 0.15% TC | 0.70 |
| Softsoap ® + 0.3% TC | 0.81 |
| Softsoap ® + 0.5% LG oil | 0.76 |
| Softsoap ® + 0.7% LG oil | 0.75 |
| Softsoap ® + 0.15% TC + 0.5% LG oil | 0.74 |
| Softsoap ® + 0.15% TC + 0.7% LG oil | 0.92 |
| Softsoap ® + 0.3% TC + 0.5% LG oil | 0.77 |
| Softsoap ® + 0.3% TC + 0.7% LG oil | 0.77 |

*Log 10 reduction from control bacterial counts (ranges from 5.8 × 10⁷ to 6.4 × 10⁷ cfu)

These results indicate that no synergistic or enhanced effect was seen when triclosan was combined with LG oil at weight ratios falling within the range of 1:1.7 to 1:4.6.

### Example 14

The antibacterial activity of soaps containing 1) TC-LGO-Cit 6 at weight ratios within the range of between 1:3.3 and 1:4.7 (TC:LG) and between 1:1.4 and 1:2 (LG:Citric acid) were evaluated against S. aureus. To prepare the soaps, triclosan/essential oil(s)/citric acid were dissolved in SDA40 B alcohol and added to Softsoap® (lacking triclosan) and diluted with water, so that the final concentration of alcohol was 5.5% and the final concentration of Softsoap® was 92 percent. A mixture of 0.1 ml of 10⁷ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hours and the colony counts were determined. The results are shown in Table 131.

TABLE 131

| Soap | Log 10 reduction from control* |
|---|---|
| Softsoap ® | — |
| Softsoap ® + 0.15% TC | 0.24 |
| Softsoap ® + 1% citric acid | 1.49 |
| Softsoap ® + 0.15% TC + \grave{ } % | 2.01 |
| citric acid | |
| Softsoap ® + 0.15% TC + 0.5% LG | 2.41 |
| oil + 1% citric acid | |
| Softsoap ® + 0.15% TC + 0.4% LG | 7.93 |
| oil + 0.2% Orange oil + 1% citric acid | |

*Log 10 reduction from control bacterial counts (ranges from 1 × 10⁶ to 5 × 10⁶ cfu).

The foregoing data show that citric acid was found to enhance the activity of triclosan, and that addition of LG oil+O oil to a combination of triclosan and citric acid further enhanced the effect.

### Example 15

The following experiments were performed to compare the antibacterial activity of combinations of (i) lemongrass oil citric acid+triclosan; (ii) lemongrass oil+citric acid; and (iii) cinnamon oil-citric acid+triclosan, all in a Softsoap® base.

To prepare the soaps, triclosan/essential oil/citric acid were dissolved in SDA40 B alcohol and added to Softsoap® (lacking triclosan) and diluted with water, so that the final concentration of alcohol was 5.5% and the final concentration of Softsoap® was 92 percent. A mixture of 0.1 ml of 10⁸ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hours and the colony counts were determined. The results are shown in Table 132.

TABLE 132

| Soap | Log 10 reduction from control* |
|---|---|
| Softsoap ® + 0.15% TC | 0.7 |
| Softsoap ® + 0.15% TC + 0.4% LG | 7.93 |
| oil + 0.2% Orange oil + 1% | |
| citric acid | |
| Softsoap ® + 0.4% LG oil + 0.2% | 5.73 |
| Orange oil + 1% citric acid | |
| Softsoap ® + 0.15% TC + 0.4% C | 5.50 |
| oil + 0.2% Orange oil + 1% | |
| citric acid | |
| Softsoap ® + 0.4% C oil + 0.2% | 4.39 |
| Orange Oil + 1% citric acid | |

*Log 10 reduction from control bacterial counts (ranges from 6.4 × 10⁷ to 9.9 × 10⁷ cfu)

The above data demonstrate, among other things, that LGO-Cit+Triclosan was found to be more effective than LGO-Cit and CO-Cit+Triclosan.

### Example 16

The following experiments were performed to evaluate the effect of adding various essential oil combinations, citric acid (0.5-0.7%), and SDA 40 B alcohol (5.5%) to commercial triclosan-containing soaps such as Dial® Soap and Softsoap®) containing 0.15% Triclosan (“Dial® Soap-TC” and “Softsoap®-TC” respectively). A mixture of 0.1 ml of 10⁸ cfu/ml of S. aureus culture and 0.1 ml of bovine serum were placed in a sterile culture tube. 0.8 ml of the test soap formulation (or phosphate buffered saline as control) was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hours and the colony counts were determined. The formulations are shown in Tables 133-136. The results are shown in Table 137.

TABLE 133
Dial ® Soap TC-CO-Cit 7

| Ingredient | Percentage (w/w) |
|---|---|
| Cinnamon oil | 0.5 |
| Orange Oil | 0.2 |
| Citric acid | 1.0 |
| SDA 40 B alcohol | 5.5 |
| Dial ® Soap-TC | 92.8 |

TABLE 134
Dial ® Soap TC-LGO-Cit 7

| Ingredient | Percentage (w/w) |
|---|---|
| Lemon Grass oil | 0.5 |
| Orange Oil | 0.2 |
| Citric acid | 1.0 |
| SDA 40 B alcohol | 5.5 |
| Dial Soap ®-TC | 92.8 |

TABLE 135
Dial ® Soap TC-LG-Cit 5

| Ingredient | Percentage (w/w) |
|---|---|
| Lemon Grass oil | 0.5 |
| Citric acid | 1.0 |
| SDA 40 B alcohol | 5.5 |
| Dial ® Soap-TC | 92.8 |

TABLE 136
Softsoap ® TC-LGO-Cit 7

| Ingredient | Percentage (w/w) |
|---|---|
| Lemon Grass oil | 0.5 |
| Orange Oil | 0.2 |
| Citric acid | 1.0 |
| SDA 40 B alcohol | 5.5 |
| Softsoap ®-TC | 92.7 |

TABLE 137
Results

| Soap | Log 10 reduction from control* |
|---|---|
| Dial ® Soap TC | 0.36 |
| Dial ® Soap-TC-CO-Cit 7 | 3.9 |
| Dial ® Soap-TC-LG-Cit 5 | 3.35 |
| Dial ® Soap-TC-LGO-Cit 7 | 5.09 |
| Softsoap ®-TC | 0.33 |
| Softsoap ®-TC-LGO-Cit 7 | 4.66 |
| Softsoap ®-TC + 1% | 2.64 |
| citric acid | |

*Log reduction from control bacterial counts (ranges from 2 0 × 10⁸ to 3.5 × 10⁸ cfu)

The above results indicate that citric acid was found to enhance the activity of soaps containing triclosan; the combination of citric acid and essential oils was found to increase the antimicrobial activity of soap containing triclosan, and superior antimicrobial action was associated with a combination of citric acid, lemongrass and orange oils, and triclosan.

### Example 17

The pH of soaps containing 1% citric acid typically ranges between 3.2-3.3. To determine whether or not the superior efficacy observed with the combination of essential oils and citric acid is due to the acidic pH, certain EO/citric acid containing-soaps were adjusted to pH 6.0 with 10 N sodium hydroxide and their antibacterial efficacy tested and compared to the corresponding soaps without pH adjustment. For the evaluation of antimicrobial activity, a mixture of 0.1 ml of 10⁷ cfu/ml of S. aureus culture (ATCC #6538) and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 138. (“Softsoap®-TC” is Softsoap® containing 0.15 percent triclosan).

TABLE 138

| Soap | Log 10 reduction from control* |
|---|---|
| CO-Cit + Softsoap ®-TC pH 3.25 | 3.9 |
| CO-Cit + Softsoap ®-TC pH 6.0 | 3.25 |
| CLGO-Cit + Softsoap ®-TC pH 3.25 | 5.1 |
| CLGO-Cit + Softsoap ®-TC pH 6.0 | 5.65 |

*Log 10 reduction from control bacterial counts (ranges from 1 × 106 to 5 × 106.)

Conclusion: The efficacy was similar at both pH values tested. This indicates that the superior activity of essential oils and citric acid observed is not due to the acidic pH.

### Example 18

Household cleansers were prepared comprising citric acid (1-2%), alcohol, and either (i) lemongrass oil; (ii) a combination of lemongrass oil and pine oil; (iii) a combination of lemongrass oil and orange oil; or (iv) a combination of pine oil and orange oil. The antimicrobial effectiveness of these formulations were tested and compared to commercial Pinesol® cleanser (containing 8.7 percent pine oil and other ingredients including detergent and other cleaning agents) as a control.

TABLE 139
Stock solution of hard surface Disinfectant-LG-Cit 2

| Ingredient | Percentage (w/w) |
|---|---|
| Lemongrass oil | 2.0 |
| Citric Acid | 20.0 |
| SDA 40B alcohol | 76.5 |
| Pluronic Surfactant L-61 | 0.5 |
| Pluronic Surfactant F-127 | 0.5 |
| Pluronic Surfactant F-87 | 0.5 |

TABLE 140
Stock solution of hard surface Disinfectant.-LGP-Cit 4

| Ingredient | Percentage (w/w) |
|---|---|
| Lemongrass oil | 1.0 |
| Pine oil | 3.0 |
| Citric Acid | 20.0 |
| SDA 40B alcohol | 74.5 |
| Pluronic Surfactant L-61 | 0.5 |
| Pluronic Surfactant F-127 | 0.5 |
| Pluronic Surfactant F-87 | 0.5 |

TABLE 141
Stock solution of hard surface Disinfectant.-P-Cit 5

| Ingredient | Percentage (w/w) |
|---|---|
| Pine oil | 5.0 |
| Citric Acid | 20.0 |
| SDA 40B alcohol | 73.5 |
| Pluronic Surfactant L-61 | 0.5 |
| Pluronic Surfactant F-127 | 0.5 |
| Pluronic Surfactant F-87 | 0.5 |

After tenfold dilution of each stock solution the disinfectant contained the following percentages (w/w) of each ingredient.

TABLE 142

| Disinfectant | Ingredients |
|---|---|
| Surface Disinfectant - LG cit 2 | 0.2% Lemon grass oil |
| | 2% Citric acid |
| | 7.65% Alcohol |
| | 0.15% surfactants |
| Surface Disinfectant - LG P cit 4 | 0.3% Pine oil |
| | 0.1% Lemon grass oil |
| | 2% Citric Acid |
| | 7.45% alcohol |
| | 0.15% Surfactants |
| Surface Disinfectant - P cit 5 | 0.5% Pine oil |
| | 2% Citric acid |
| | 7.45% alcohol |
| | 0.15% surfactants |

To prepare the solution of Pinesol® to serve as control, as per the manufacturer's instruction, 6 ml of the Pinesol® containing 8.5% pine oil was diluted to 100 ml. This diluted sample contained 0.52% pine oil.

To test the antimicrobial activity, 0.1 ml of culture containing approximately 1×10⁷ colony forming units (“cfu”) of S. aureus per milliliter was spread evenly on the surface of 2.5×11 cm² tiles using a glass rod and left at room temperature for 10 minutes to dry. After 10 minutes 0.3 ml of the diluted surface disinfectant was spread evenly on the tiles with a sterile glass rod and left for another 10 minutes to dry. The tiles were rinsed with 9.6 ml of inactivating medium (BPBNS), which was collected for testing. The collected medium was serially diluted and 0.5 ml was plated onto TSA plates and incubated at 37° C. for 18-24 hours. The colonies on the plates were counted and the values converted to log₁₀.

TABLE 143

| | Log₁₀ reduction from control bacterial counts* | | | |
|---|---|---|---|---|
| | Disinfectant | Disinfectant | Disinfectant | |
| Organism | LG-Cit | LGP-Cit | P-Cit | Pine Sol |
| S. aureus | 3.56 | 1.89 | 0.81 | 2.4 |

*Log 10 reduction from control bacterial counts (ranges from 1 × 10⁶-5 × 10⁶)

These data indicate that a surface cleaner containing 0.2% LG oil and 2.0% Citric acid was found to be considerably more effective than a cleaner containing 0.5% Pine oil and 2% Citric acid as well as commercial Pinesol® Surface cleaner containing 0.52% Pine oil. The cleanser containing 0.3% Pine oil+0.1% LG oil+2% Citric acid was also found to be more effective than the one containing 0.5% Pine oil and 2% Citric acid.

### Example 19

The following stock solution was prepared.

TABLE 144
Stock Solution of hard surface Disinfectant. - POCit 7

| Ingredient | Percentage (w/w) |
|---|---|
| Pine oil | 5.0 |
| Orange oil | 2.0 |
| Citric Acid | 10.0 |
| SDA 40B alcohol | 53.5 |
| Pluronic Surfactant L-61 | 0.5 |
| Pluronic Surfactant F-127 | 0.5 |
| Pluronic Surfactant F-87 | 0.5 |

7.2% of the stock hard disinfectant was diluted with water to 100% before use. These diluted samples contained the following concentrations of active ingredients.

TABLE 145

| Disinfectant | Ingredients |
|---|---|
| Surface Disinfectant - PO Cit 7 | 0.5% Pine oil |
| | 0.2% Orange oil |
| | 1% Citric Acid |
| | 5.35% alcohol |
| | 0.15% Surfactants |

The following stock solution was prepared:

TABLE 146
Stock Solution of hard surface Disinfectant. - LGOCit 7

| Ingredient | Percentage (w/w) |
|---|---|
| Lemongrass oil | 5.0 |
| Orange oil | 2.0 |
| Citric Acid | 10.0 |
| SDA 40B alcohol | 53.5 |
| Pluronic Surfactant L-61 | 0.5 |
| Pluronic Surfactant F-127 | 0.5 |
| Pluronic Surfactant F-87 | 0.5 |

7.2% of the stock hard disinfectant was diluted with water to 100% before use. This diluted samples contained the following concentrations of active ingredients:

TABLE 147

| Disinfectant | Ingredients |
|---|---|
| Surface Disinfectant - LGO Cit 7 | 0.5% LG oil |
| | 0.2% Orange oil |
| | 1% Citric Acid |
| | 5.35% alcohol |
| | 0.15% Surfactants |

The method used in Example 18 was used to test antimicrobial activity.

TABLE 148

| | S. aureus | P. aeruginosa | E. coli |
|---|---|---|---|
| Log 10 reduction in | 0.6 | 5.1 | 5.1 |
| bacteria - PO-Cit 7 | | | |
| Log10 reduction in | 5.9 | 4.8 | 5.09 |
| bacteria - LGO-Cit 7 | | | |

*Log₁₀ reduction from control bacterial counts (ranges from 1 × 10⁶-5 × 10⁶)

The foregoing data indicate that LGO-Cit is effective against both gram positive and gram negative organisms while PO Cit is not very effective against the Gram positive organism S aureus.

### Example 20

The following experiments were carried out using either soap or surface disinfectants containing the EO(s)/citric acid combinations indicated. The test organism used was Candida albicans.

Where soap was employed, the following method was used. A mixture of 0.1 ml of 10⁷ cfu/ml of C. albicans culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results, showing 30 second kill activity, are shown in Table 149.

Where surface disinfectant was employed, the following method was used. 0.1 ml of culture containing approximately 1×10⁷ colony forming units (“cfu”) of C. albicans per milliliter was spread evenly on the surface of 2.5×11 cm² tiles using a glass rod and left at room temperature for 10 minutes to dry. After 10 minutes 0.3 ml of the diluted surface disinfectant was spread evenly on the tiles with a sterile glass rod and left for another 10 minutes to dry. The tiles were rinsed with 9.6 ml of inactivating medium (BPBNS), which was collected for testing. The collected medium was serially diluted and 0.5 ml was plated onto TSA plates and incubated at 37° C. for 18-24 hours. The colonies on the plates were counted and the values converted to log₁₀.

TABLE 149

| Formulation | Log10 reduction from control* |
|---|---|
| CO-Cit 6 Soap | 1.02 |
| LGO-Cit 6 Soap | 1.27 |
| CO-Cit 7 Surface Disinfectant | 5.2 |
| LGO-Cit 7 Surface Disinfectant | 4.81 |

*Control counts range from 1 × 10⁶ to 5 × 10⁶

These results show that CO groups and LGO groups show similar activity against C. albicans.

### Example 21

Evaluation of the rapid antibacterial activity of various soap formulations was performed as follows.

Method of evaluation of rapidity of kill of soaps. The rapid antimicrobial efficacy of the soaps containing LG and various combinations were tested as follows. A mixture of 0.1 ml of 10⁹ cfu/ml of bacterial cultures and 0.1 ml of bovine serum was placed in a sterile culture tube. 0.8 ml of the test soap formulation was added to the tube and vortexed for 30 seconds. 9.0 ml drug neutralizing fluid (DNF) was added to the tube to neutralize the activity of the soap, this tube was vortexed and serially diluted with DNF. 0.5 ml of the diluted solution was plated on trypticase soy agar plates, incubated at 37° C. for 24-48 hours and the colony counts were determined. The soap base without essential oils citric acid, secondary alcohol and Incroquat containing the culture were also tested. PBS was used as the control. LG-O-Cit 5 comprises 0.3 percent (weight/weight) lemongrass oil, 0.3 percent (weight/weight) orange oil, 1.0 percent (weight/weight) citric acid, 1.0 percent (weight/weight) 2-phenoxyethanol and 15 percent (weight/weight) SDA-40B alcohol. LG-O-Cit 4 comprises 0.3 percent (weight/weight) lemongrass oil, 0.1 percent (weight/weight) orange oil, 1.0 percent (weight/weight) citric acid, 1.0 percent (weight/weight) 2-phenoxyethanol and 15 percent (weight/weight) SDA-40B alcohol. The amount of alkanediol, where present, is 0.3 percent (weight/weight). The complete formulations for the soaps specified are set forth in section 4.9, above. The results are shown in Table 150 below.

TABLE 150
Enhancement of the antibacterial activity of LG-O-Cit composition
by 0.3% of alkanediols (Test Organism: S. aureus)

| Soap formulations | Log10 reduction from control |
|---|---|
| Base | 1.8 |
| LG-O-Cit 5 | 3.7 |
| 1,2 decanediol (0.3%) | 0.6 |
| LG-O-Cit 5 + 1,2 decanediol | 4.5 |
| LG-O-Cit 4 | 3.6 |
| LG-O-Cit 4 + 1,2 decanediol | 4.8 |
| LG-O-Cit 4 + 1,2 dodecanediol | 4.5 |
| LG-O-Cit 4 + 1,2 Tetradecanediol | 4.5 |

*Log₁₀ reduction from Control bacterial counts (ranges from 2 × 10⁸-5 × 10⁸)

The results shown in Table 150 indicate that the alkanediols tested enhanced the antibacterial activity of LG and O oil and citric acid disinfectant composition at a concentration of 0.3 percent (weight/weight).

### Example 22

The method described in Example 21, above, was used to evaluate the antibacterial activity of soap formulations comprising 0.5 percent of alkanediols. LG-O-Cit 4A comprises 0.3 percent (weight/weight) lemongrass oil, 0.1 percent (weight/weight) orange oil, 1.0 percent (weight/weight) citric acid, 1.0 percent (weight/weight) 2-phenoxyethanol and 17 percent (weight/weight) SDA-40B alcohol. The amount of alkanediol, where present, is 0.5 percent (weight/weight). The complete formulations for the soaps specified are set forth in section 4.6, above. The results are shown in Table 151 below.

TABLE 151
Enhancement of the antibacterial activity of LG-O-Cit A
Composition by 0.5% of alkanediols
Rapid antimicrobial activity (30 second Kill)
(Test Organism S. aureus)

| Soap formulations | Log10 reduction from control |
|---|---|
| Base | 0.8 |
| LG-O-Cit 4A | 4.1 |
| 1,2 decanediol(0.5%) | 1.4 |
| LG-O-Cit 4A + 1,2 decanediol | 6.0 |
| LG-O-Cit 4A + 1,2 dodecanediol | 6.1 |
| LG-O-Cit 4A + 1,12 dodecanediol | 6.0 |
| LG-O-Cit 4A + 1,2 Tetradecanediol | 6.0 |
| LG-O-Cit4A + 0.25% 1,2decanediol + | 6.0 |
| 0.25% 1,12Dodecanediol | |
| Cn-O-Cit4A | 3.7 |
| Cn-O-Cit4A + 1,2 decanediol | 4.9 |

(pH of all the soaps ranged from 4.5-4.6)
*Log₁₀ reduction from Control bacterial counts (ranges from 2 × 10⁸-5 × 10⁸)

The results shown in Table 151 indicate that alkanediols at 0.5% concentration showed significant enhancement of the antibacterial activity of LG+O oil+citric acid or Cn+O oil and citric acid disinfectant composition.

### Example 23

To evaluate the effect of decanediol on the antibacterial activity of citric acid or citric acid in combination with essential oils, the following experiments were performed. The compounds indicated below were incorporated into soft soap lacking triclosan and the activity was evaluated. Activity was measured as described in Example 22. The results are shown in Table 152.

TABLE 152
Rapid antimicrobial activity (30 second Kill)
(Test Organism S. aureus)

| | Log₁₀ reduction |
|---|---|
| Soap formulations (% w/w) | from control |
| Plain Soft soap | 0.2 |
| 0.5 decanediol | 1.4 |
| 1.0 citric acid | 1.3 |
| 0.5 decanediol + 1 citric acid | 6.5 |
| 0.3 + 0.1 LG + O | 0.1 |
| 0.5 decanediol + 1 citric acid + 0.3 + 0.1 LG + O | 7.0 |
| 0.25 decanediol + 0.5 citric | 4.7 |
| 0.15 + 0.06 LG + O | 0.1 |
| 0.25 decanediol + 0.5 citric + 0.15 + 0.06 LG + O | 5.6 |

*Log₁₀ reduction from PBS(Control) bacterial counts (ranges from 7 × 10⁷-1 × 10⁸)

The results shown in Table 152 indicate that decanediol and citric acid exhibit synergistic activity, and that further addition of essential oil enhances the activity. The use of decanediol+citric acid+essential oils in soap even at low concentrations was found to show superior antibacterial activity.

### Example 24

To determine the effect of LG-O-Cit-1,2 decanediol on the antibacterial activity of triclosan-containing soap, the following experiments were performed.

Dial® soap containing 0.15% Triclosan (Dial-T Soap) was used for this test. The following formulation was prepared. The antibacterial activity was then tested using the method set forth in Example 21. The results are shown in Table 153.

TABLE 153
Dial ®-T Soap Containing LG-O-Cit 4 and 0.5% 1,2 decanediol

| Ingredient | Percentage (w/w) |
|---|---|
| Dial ® - T soap | 90.0 |
| SDA 40B | 8.1 |
| Lemon grass oil | 0.3 |
| Orange oil | 0.1 |
| Citric acid | 1.0 |
| 1,2 decanediol | 0.5 |

Original pH was 3.2 pH adjusted to 4.5 with 10.N NaOH.

TABLE 154
Enhancement of the activity of Triclosan by LG-O-Cit-1,2 Decanediol
Rapid antimicrobial activity (30 second Kill)
(Test Organism S. aureus)

| | Log₁₀ reduction |
|---|---|
| Soap formulations | from control |
| Dial ®-T soap | 0.7 |
| Dial ®-T Soap + LG-O-Cit 4 | 5.5 |
| Dial ®-T Soap + LG-O-Cit4-0.5% 1,2 decanediol | 8.0 |

*Log₁₀ reduction from Control bacterial counts (ranges from 2 × 10⁸-5 × 10⁸)

The foregoing results indicate that decanediol enhances the activity of Dial®-T Soap+LG-O-Cit 4.

### Example 25

The antibacterial activity of LG-O-CitA-D-T Lotion, having the following formulation, was tested in a pigskin model.

TABLE 155

| Ingredient | Percentage (w/w) |
|---|---|
| Water | 65.6 |
| U Care-JR 30M | 0.25 |
| PolyoxWSR-205 | 0.1 |
| Incroquat TMS Behenyl | 2.0 |
| Isopropyl myristate | 1.0 |
| Acetulan | 1.0 |
| Vitamin E | 0.2 |
| Zinc stearate | 0.2 |
| Polawax NF | 2.75 |
| Glycerin | 2.0 |
| Allantoin | 0.2 |
| Dimethicone copolyol (Q2-5220) | 2.5 |
| Citric acid | 1.0 |
| 1,2 decanediol | 0.5 |
| Tocopheryl acetate | 0.5 |
| Glyceryl stearate (Arlacel165) | 1.0 |
| Butylene glycol | 3.0 |
| SDA-40-B | 15 |
| Lemongrass oil | 0.5 |
| Tea tree oil | 0.5 |
| Orange oil | 0.1 |
| 1,2 Decanediol(Symclairol) | 0.5 |
| Triclosan | 0.3 |

(pH adjusted to 4.5-5.0)

The pigskin model assay was as follows. Six sets of 3×3 cm² pig skin each mounted on a petriplate were rinsed in 70% isopropanol, and air dried. One piece of the pair was contaminated with 30 μl of 10⁸ cfu of MRSA culture; the two pieces were then rubbed against each other for 30 seconds, and left at 37° C. to dry for one hour. 3 pairs were used for control and another 3 pairs were used for the test, which was as follows.

To one piece of the pair from the control, 0.1 gm of placebo cream same as LG-O-Cit4-D (above) without SDA-40-B, lemongrass oil, tea tree oil, orange oil, 1,2 decanediol (Symclairol) was applied, and rubbed against the other piece for 15 seconds and left at 37° C. for 1 hour. The same procedure was repeated with the test skins in which LG-O-CitA-D-T was applied. Following this, 0.2 ml dilution media (DM) was added to one skin piece and both pieces rubbed again for 15 seconds. The surviving organisms were recovered from the skin by rinsing each piece with 9.9 ml of DM. The washing fluid from both pieces was collected in one petri dish, mixed and transferred to a culture tube from which further serial dilutions were made. Aliquots from the dilutions were plated on TSA plates and incubated for 24-48 hours at 37° C. before colony counts (baseline counts) were determined. The results are shown in Table 156.

TABLE 156
Reduction of Bacterial growth 1 hour post treatment

| | Bacterial counts | Log₁₀ reduction from |
|---|---|---|
| Treatment cream | (cfu/skin) | control counts |
| PBS | 2.2 × 10⁶ | — |
| Placebo cream(control) | 2.0 × 10⁶ | — |
| LG-O-Cit A-D - T Lotion | 7.6 × 10³ | 2.37 |

### Example 26

The antibacterial activity of preservative compositions was evaluated.

TABLE 157
Preservative composition A

| Ingredient | Percentage (w/w) |
|---|---|
| Lemongrass oil | 10 |
| Farnesol | 10 |
| Orange oil | 5 |
| lactic acid | 7 |
| 1,2 decanediol | 7 |
| SDA 40 B alcohol | 61 |

TABLE 158
Preservative composition B

| Ingredient | Percentage (w/w) |
|---|---|
| Lemongrass oil | 15 |
| Farnesol | 15 |
| Orange oil | 10 |
| lactic acid | 10 |
| SDA 40 B alcohol | 50 |

TABLE 159
Preservative composition C

| Ingredient | Percentage (w/w) |
|---|---|
| Farnesol | 17 |
| Citric acid | 7 |
| 1,2 decanediol | 7 |
| SDA 40 B alcohol | 69 |

TABLE 160
Preservative composition D

| Ingredient | Percentage (w/w) |
|---|---|
| Lemongrass oil | 15 |
| Orange oil | 5 |
| Lactic acid | 10 |
| 1,2 decanediol | 20 |
| 1,2 Octanediol | 20 |
| SDA 40 B alcohol | 30 |

TABLE 161
Preservative composition E

| Ingredient | Percentage (w/w) |
|---|---|
| Lemongrass oil | 15 |
| Orange oil | 5 |
| Lactic acid | 10 |
| 1,2 Octanediol | 40 |
| SDA 40 B alcohol | 30 |

The pH of these solutions are adjusted to 5.0. 0.5-5.0% of these preservatives can be used in various formulations.

Evaluation of the Preservative efficacy of Composition A and B. The following Cream base was prepared to incorporate the preservative before testing.

TABLE 162
Preservative composition F

| Ingredient | Percentage (w/w) |
|---|---|
| Water | 70.24 |
| Ucare JR 40 | 0.3 |
| Polowax | 3.0 |
| Incroquat Behenyl TMS | 3.0 |
| Petroleum jelly | 5.0 |
| Stearyl alcohol | 7.0 |
| Propylene glycol | 2.0 |
| Isopropyl myristate | 4.0 |
| Sorbitan oleate | 2.0 |
| Polyoxyl 40 stearate | 2.0 |

An overnight culture of bacteria grown in Trypticase Soy Broth (TSB) was diluted with TSB to obtain 10⁸ CFU organism/ml. For the test samples, 2% of the preservative was added to 10 grams of the cream and mixed well. From this sample, 1 gram aliquots were placed into 10 ml sterile plastic culture tubes and 0.1 ml (100 microliters) of the test inoculum was added and vortexed until uniformly blended. The tubes were then placed into incubators at 37° C. All tubes were incubated for a total of 3 days. At the end of the incubation period 9.0 ml of Butterfield Phosphate Buffered solution with neutralizer was added to the incubated cultured sample and vortexed until completely mixed. The samples were serially diluted and then plated in Trypticase soy agar (TSA). the plates were incubated at 37° C. temperature for 24-48 hours and the counts were read. The results are shown in Table 163, below.

TABLE 163
Log₁₀ Reduction from control growth

| | S. aureus | P. aeruginosa |
|---|---|---|
| Control | — | — |
| Preserv A | 7.8 | 8.0 |
| Preserv B | 6.7 | 4.0 |

Control growth for S. aureus and P aeruginosa are 6.5×10⁸ and 1×10⁸ cfu/gm respectively.

### Example 27

The following experiments were performed to evaluate wound dressings impregnated with essential oils, citric acid and decanediol.

TABLE 164
Antimicrobial Impregnation solution

| Ingredient | Percentage (w/w) |
|---|---|
| Lemongrass oil | 0.3 |
| Orange oil | 0.1 |
| Tea tree oil | 0.5 |
| Calandula oil | 0.5 |
| Citric acid | 1.0 |
| Olive oil | 5.0 |
| Propylene glycol | 10 |
| Decanediol | 0.5 |
| SDA 40 B alcohol | 51.7 |
| U care JR 30 | 0.4 |
| Water | 30 |

TABLE 165
Antimicrobial/anti inflammatory Impregnation solution

| Ingredient | Percentage (w/w) |
|---|---|
| Lemongrass oil | 0.3 |
| Orange oil | 0.1 |
| Tea tree oil | 0.5 |
| Calandula oil | 0.5 |
| Citric acid | 1.0 |
| Olive oil | 5.0 |
| Propylene glycol | 10 |
| Decanediol | 0.5 |
| SDA 40 B alcohol | 51.0 |
| U care JR 30 | 0.4 |
| Curcumin | 0.3 |
| Water | 29.7 |

Wound dressings (Dukal non adherent pad) were dipped into the antimicrobial impregnation solution and dried for 24 hours. The dressings were cut into 1 cm² and the zones of inhibition against various organisms were determined.

Zones of inhibition test. 1×1 cm² piece of each dressing was placed on Trypticase soy agar plate seeded on the surface with 0.3 mL of 10⁸ colony forming units (CFU)/mL) of the test organism. The plates were incubated at 37° C. for 24 hours. The zone of inhibition around the catheter segments, excluding the diameter of patch was measured. The results are shown in Table 166.

TABLE 166
Antimicrobial Impregnation solution

| Organism | Zone of inhibition (mm) |
|---|---|
| S. aureus | 7.0 |
| MRSA | 8.0 |
| P. aeruginosa | 5.0 |
| C. albicans | 9.0 |

### Example 28

The following experiment was performed to evaluate the efficacy of creams containing preservative compositions.

Creams containing 2.0%-3.0% of preservative compositions were prepared and tested according to the following method.

TABLE 167
Formulation

| Ingredients | Percentage (w/w) |
|---|---|
| Water | 70.24 |
| Ucare JR 40 | 0.3 |
| Polowax | 3.0 |
| Incroquat Behenyl TMS | 3.0 |
| Petroleum Jelly | 5.0 |
| Stearyl alcohol | 7.0 |
| Propylene glycol | 2.0 |
| Isopropyl myristate | 4.0 |
| Sorbitan oleate | 2.0 |
| Polyoxyl 40 stearate | 2.0 |

An overnight culture of bacteria grown in Trypticase Soy Broth (TSB) was diluted with TSB to obtain 10⁸ CFU organism/ml. For the test samples, 2% of the preservative was added to 10 grams of the cream and mixed well. From this sample, 1 gram aliquots were placed into 10 ml sterile plastic culture tubes and 0.1 ml (100 microliters) of the test inoculum was added and vortexed until uniformly blended. The tubes were then placed into incubators under the following temperatures: 30° C. for Aspergillus niger and 37° C. for the remaining three microbes. All tubes were incubated for a total of 3 days. At the end of the incubation period, 9.0 ml of Butterfield Phosphate Buffered solution with neutralizer was added to the incubated cultured sample and vortexed until completely mixed. The samples were serially diluted and then plated in Trypticase soy agar (TSA). The plates were incubated at 37° C. temperature for 24-48 hours, and the counts were read. Placebo cream was tested similarly and used as the control. The following Table reflects the results of the testing.

TABLE 168
Log10 Reduction from control growth

| Preservative | S. aureus | P. aeruginosa | Aspergillus niger |
|---|---|---|---|
| F | 6.8 | 4.65 | 5.0 |
| G | 7.3 | 5.8 | 5.1 |
| H | 5.5 | 5.0 | 3.0 |
| I | 6.5 | 5.0 | 4.9 |
| J | 5.5 | 4.5 | 3.0 |
| K | 5.8 | 4.8 | 4.0 |
| L | 6.0 | 5.2 | 3.8 |
| M | 6.8 | 5.5 | 5.0 |

Control growth for S. aureus and P aeruginosa were 6.5×10⁸ and 1×10⁸ cfu/gm, respectively, and for A. niger was 6×10⁴-1×10⁵. Based on these results, all of the above preservative compositions were effective.

### Example 29

The following experiment was carried out to evaluate the synergistic effect of botanical extract, essential oil and fruit acids in a soap base.

Antibacterial efficacy of Grape fruit seed extract (GFSE) either alone or in combination with citric acid was determined by Method A (discussed below) and the results are shown in Table 169. Citric acid alone or a mixture of fruit acids such as lactic, citric, tartaric, glycolic and malic (Multi Fruit® BSC from Arch chemicals) were used.

Method A: 0.8 gms of plain Softsoap containing the following combinations (Table 169) was mixed with 0.1 ml of S. aureus culture (10⁸ cfu/ml) and 0.1 ml bovine serum. After 30 seconds, 9 ml of drug neutralizing media(DNB) was added and mixed. Then, serial dilutions were made with DNB and plated on Trypticase Soy agar. Table 169 shows the results of the Log reduction from control counts.

TABLE 169
Log reduction from control counts

| Group | (% w/w) | Log reduction |
|---|---|---|
| GSE | 0.3 | 0.87 |
| citric acid | 1.0 | 0.7 |
| GSE + citric | 0.3 + 1.0 | 3.28 |
| Lemon Grass oil (LG) | 0.2 | 0.04 |
| LG + Citric | 0.2 + 1.0 | 1.5 |
| GSE + LG + citric | 0.3 + 0.2 + 1.0 | 5.35 |
| Lactic acid | 0.2 | 0.5 |
| GSE + Lactic acid | 0.3 + 0.2 | 3.2 |

### Example 30

The following example demonstrates preservative compositions containing low concentrations of essential oil/botanical extract.

The following preservative compositions were prepared and tested for their effectiveness. The preservative compositions contain the following: total Essential oil/botanical extracts concentration ranges from 1.0-20%, fruit acids ranging from 10-20%, alkanediols ranging from 30-80%, and alcohol ranging from 0-40%, phenoxy ethanol ranging from 0-40%, propylene glycol ranging from 0-80%, and vegetable oil ranging from 0-50%. Tables 170-179 provide the formulations of specific preservative compositions with essential oil and fruit acids.

TABLE 170
Preservative composition-1

| | | Products containing 1.75% |
|---|---|---|
| Ingredients | Stock (% w/w) | stock (% w/w) |
| Lemongrass oil | 5.6 | .01 |
| Orange oil | 2.8 | 0.05 |
| Lactic acid | 11.4 | 0.2 |
| Octanediol | 40 | 0.7 |
| Phenoxyethanol | 40 | 0.7 |
| (pH of Stock solution 5.0) | | |

TABLE 171
Preservative composition-2

| | Stock | Products containing 2.0% |
|---|---|---|
| Ingredients | (% w/w) | stock (% w/w) |
| Lemongrass oil | 3.75 | 0.075 |
| Orange oil | 1.25 | 0.025 |
| Lactic acid | 15 | 3.0 |
| Octanediol | 40 | 0.8 |
| Phenoxyethanol | 40 | 0.8 |
| (pH of Stock solution 5.0) | | |

TABLE 172
Preservative composition -3

| | | Products containing 2.0% |
|---|---|---|
| Ingredients | Stock (% w/w) | stock (% w/w) |
| Lemongrass oil | 2.0 | 0.04 |
| Orange oil | 0.5 | 0.01 |
| Lactic acid | 10 | 0.2 |
| Octanediol | 60 | 1.2 |
| Phenoxyethanol | 27.5 | 0.55 |
| (pH of Stock solution 5.0) | | |

TABLE 173
Preservative composition -4

| | | Products containing 2.0% |
|---|---|---|
| Ingredients | Stock (% w/w) | stock (% w/w) |
| Lemongrass oil | 5 | 0.1 |
| Orange oil | 2.5 | 0.05 |
| Lactic acid | 10 | 0.2 |
| Octanediol | 40 | 0.8 |
| Propylene glycol | 42.5 | 0.8 |
| (pH of Stock solution 5.0) | | |

TABLE 174
Preservative composition -5

| | | Products containing 2.0% |
|---|---|---|
| Ingredients | Stock (% w/w) | stock (% w/w) |
| Lemongrass oil | 5 | 0.1 |
| Orange oil | 2.5 | 0.05 |
| Lactic acid | 10 | 0.2 |
| Octanediol | 50 | 1.0 |
| Propylene glycol | 32.5 | 0.65 |
| (pH of Stock solution 5.0) | | |

TABLE 175
Preservative composition -6

| | | Products containing 2.0% |
|---|---|---|
| Ingredients | Stock (% w/w) | stock (% w/w) |
| Lemongrass oil | 3.75 | 0.075 |
| Orange oil | 1.25 | 0.025 |
| Lactic acid | 10 | 0.2 |
| Octanediol | 50 | 1.0 |
| Propylene glycol | 35 | 0.7 |
| (pH of Stock solution 5.0) | | |

TABLE 176
Preservative composition -7

| | | Products containing 2.0% |
|---|---|---|
| Ingredients | Stock (% w/w) | stock (% w/w) |
| Lemongrass oil | 2.5 | 0.05 |
| Orange oil | 1.25 | 0.025 |
| Lactic acid | 10 | 0.2 |
| Octanediol | 50 | 1.0 |
| Propylene glycol | 36.25 | 0.725 |
| (pH of Stock solution 5.0) | | |

TABLE 177
Preservative composition -7-A

| | | Products containing 2.0% |
|---|---|---|
| Ingredients | Stock (% w/w) | stock (% w/w) |
| Lemongrass oil | 2.0 | 0.05 |
| Orange oil | 1.0 | 0.025 |
| Lactic acid | 8.0 | 0.2 |
| Octanediol | 40 | 1.0 |
| Propylene glycol | 49 | 1.225 |
| (pH of Stock solution 5.0) | | |

TABLE 178
Preservative composition -7-B-L

| | | Products containing 2.0% |
|---|---|---|
| Ingredients | Stock (% w/w) | stock (% w/w) |
| Lemongrass oil | 2.8 | 0.07 |
| Lactic acid | 8.0 | 0.2 |
| Octanediol | 40 | 1.0 |
| Propylene glycol | 49.2 | 1.23 |
| (pH of Stock solution 5.0) | | |

TABLE 179
Preservative composition -7-B-M

| | Stock | Products containing 2.0% |
|---|---|---|
| Ingredients | (% w/w) | stock (% w/w) |
| Lemongrass oil | 2.8 | 0.07 |
| Multifruit BSC | 8.0 | 0.2 |
| * Mixture of lactic, citric, tartaric, | | |
| glycolic, malic acid extracted from | | |
| plants (obtained from Arch | | |
| Chemicals) | | |
| Octanediol | 40 | 1.0 |
| Propylene glycol | 49.2 | 1.23 |
| (pH of Stock solution 5.0) | | |

Tables 180-192 provide the formulations of specific preservative compositions containing grapefruit seed extract and grape seed extract.

TABLE 180
Preservative composition -G-8

| | | Products containing 2.0% |
|---|---|---|
| Ingredients | Stock (% w/w) | stock (% w/w) |
| Grapefruit seed extract | 10 | 0.2 |
| Lactic acid | 10 | 0.2 |
| Octanediol | 50 | 1.0 |
| Propylene glycol | 30 | 0.6 |
| (pH of Stock solution 5.0) | | |

TABLE 181
Preservative composition -G-9

| | | Products containing 2.0% |
|---|---|---|
| Ingredients | Stock (% w/w) | stock (% w/w) |
| Grapefruit seed extract | 10 | 0.2 |
| Lactic acid | 10 | 0.2 |
| Octanediol | 50 | 1.0 |
| Safflower oil | 30 | 0.6 |
| (pH of Stock solution 5.0) | | |

TABLE 182
Preservative composition -G-10

| | | Products containing 2.0% |
|---|---|---|
| Ingredients | Stock (% w/w) | stock (% w/w) |
| Lemongrass oil | 2.5 | 0.05 |
| Orange oil | 1.25 | 0.025 |
| Grape seed Extract | 10 | 0.2 |
| Lactic acid | 10 | 0.2 |
| Octanediol | 50 | 1.0 |
| Propylene glycol | 26.25 | 0.525 |
| (pH of Stock solution 5.0) | | |

TABLE 183
Preservative composition -G-10-A

| | | Products containing 2.0% |
|---|---|---|
| Ingredients | Stock (% w/w) | stock (% w/w) |
| Lemongrass oil | 2.0 | 0.05 |
| Grape seed Extract | 8.0 | 0.2 |
| Lactic acid | 8.0 | 0.2 |
| Octanediol | 40 | 1.0 |
| Propylene glycol | 42.0 | 1.05 |
| (pH of Stock solution 5.0) | | |

TABLE 184
Preservative composition -G-10-M

| | Stock | Products containing 2.0% |
|---|---|---|
| Ingredients | (% w/w) | stock (% w/w) |
| Lemongrass oil | 2.5 | 0.05 |
| Orange Oil | 1.25 | 0.025 |
| Grape seed Extract | 10 | 0.2 |
| Multifruit BSC | 10 | 0.2 |
| * Mixture of lactic, citric, tartaric, | | |
| glycolic, malic acid extracted from | | |
| plants(obtained from Arch | | |
| Chemicals) | | |
| Octanediol | 50 | 1.0 |
| Propylene glycol | 26.25 | 0.525 |
| (pH of Stock solution 5.0) | | |

TABLE 185
Preservative composition -G-10-C

| | | Products containing 2.0% |
|---|---|---|
| Ingredients | Stock (% w/w) | stock (% w/w) |
| Lemongrass oil | 2.5 | 0.05 |
| Orange Oil | 1.25 | 0.025 |
| Grape seed Extract | 10 | 0.2 |
| Citric acid | 10 | 0.2 |
| Octanediol | 50 | 1.0 |
| Propylene glycol | 26.25 | 0.525 |
| (pH of Stock solution 5.0) | | |

TABLE 186
Preservative composition -G-11

| | | Products containing 2.0% |
|---|---|---|
| Ingredients | Stock (% w/w) | stock (% w/w) |
| Lemongrass oil | 3.75 | 0.075 |
| Orange Oil | 1.25 | 0.025 |
| Grape seed Extract | 10 | 0.2 |
| Lactic Acid | 10 | 0.2 |
| Octanediol | 50 | 1.0 |
| Propylene glycol | 25 | 0.5 |
| (pH of Stock solution 5.0) | | |

TABLE 187
Preservative composition -G-12

| | | Products containing 2.0% |
|---|---|---|
| Ingredients | Stock (% w/w) | stock (% w/w) |
| Grape seed Extract | 15 | 0.3 |
| Lactic acid | 10 | 0.2 |
| Octanediol | 50 | 1.0 |
| Propylene glycol | 25 | 0.5 |
| (pH of Stock solution 5.0) | | |

TABLE 188
Preservative composition -G-13

| | | Products containing 2.0% |
|---|---|---|
| Ingredients | Stock (% w/w) | stock (% w/w) |
| Lemongrass oil | 12 | 0.3 |
| Orange Oil | 4 | 0.1 |
| Grape seed Extract | 8.0 | 0.2 |
| Lactic Acid | 8.0 | 0.2 |
| Octanediol | 40 | 1.0 |
| Propylene glycol | 28 | 0.7 |
| (pH of Stock solution 5.0) | | |

TABLE 189
Preservative composition -G-14

| | | Products containing 2.0% |
|---|---|---|
| Ingredients | Stock (% w/w) | stock (% w/w) |
| Lemongrass oil | 0.8 | 0.02 |
| Orange Oil | 0.4 | 0.01 |
| Grape seed Extract | 12 | 0.3 |
| Lactic Acid | 8.0 | 0.2 |
| Octanediol | 40 | 1.0 |
| Propylene glycol | 38.8 | 0.97 |
| (pH of Stock solution 5.0) | | |

TABLE 190
Preservative composition -G-15

| | | Products containing 2.0% |
|---|---|---|
| Ingredients | Stock (% w/w) | stock (% w/w) |
| Lemongrass oil | 0.8 | 0.075 |
| Grape seed Extract | 12 | 0.2 |
| Lactic acid | 8.0 | 0.2 |
| Octanediol | 40 | 1.0 |
| Propylene glycol | 41.2 | 1.03 |
| (pH of Stock solution 5.0) | | |

TABLE 191
Preservative composition -G-16

| | | Products containing 2.5% |
|---|---|---|
| Ingredients | Stock (% w/w) | stock (% w/w) |
| Lemongrass oil | 2.8 | 0.07 |
| Grape seed Extract | 8.0 | 0.2 |
| Lactic Acid | 8.0 | 0.2 |
| Octanediol | 28 | 0.7 |
| Propylene glycol | 53.2 | 1.33 |
| (pH of Stock solution 5.0) | | |

TABLE 192
Preservative composition -G-17

| | | Products containing 2.5% |
|---|---|---|
| Ingredients | Stock (% w/w) | stock (% w/w) |
| Lemongrass oil | 2.8 | 0.07 |
| Grape seed Extract | 8 | 0.2 |
| Lactic Acid | 8 | 0.2 |
| Octanediol | 40 | 1.0 |
| Propylene glycol | 41.2 | 1.03 |
| (pH of Stock solution 5.0) | | |

Tables 193-198 provide the formulations of specific preservative compositions containing essential oils/botanical extracts, fruit acids and alkanediol without solvents. All of the compositions ending in “L” are noted as the L series, which contain lactic acid. All of the compositions ending in “M” are noted as the M series, which contain Multifruit®BSC. Multifruit®BSC contains a mixture of lactic, citric, tartaric, glycolic, and malic acid extracted from plants (obtained from Arch Chemicals). The pH of all the preservative compositions in Tables 193-198 were adjusted to 5.0 with 10 N. NaoH (30-70 ul/ml were required).

TABLE 193
Preservative composition -6-L

| Ingredients | Stock (% w/w) | Cream containing 1.3% stock |
|---|---|---|
| Lemongrass oil | 5.78 | 0.075 |
| Orange oil | 1.92 | 0.025 |
| Lactic Acid | 15.4 | 0.2 |
| Octanediol | 76.9 | 1.0 |

TABLE 194
Preservative composition -6-M

| Ingredients | Stock (% w/w) | Cream containing 1.3% stock |
|---|---|---|
| Lemongrass oil | 5.78 | 0.075 |
| Orange oil | 1.92 | 0.025 |
| Multifruit extract | 15.4 | 0.2 |
| Octanediol | 76.9 | 1.0 |

TABLE 195
Preservative composition -10-G-L

| Ingredients | Stock (% w/w) | Cream containing 1.5% stock |
|---|---|---|
| Lemongrass oil | 3.3 | 0.05 |
| Orange oil | 1.6 | 0.025 |
| Grapefruit seed extract | 15.0 | 0.225 |
| Lactic Acid | 13.3 | 0.2 |
| Octanediol | 66.8 | 1.0 |

TABLE 196
Preservative composition -10-G-M

| | | Cream containing |
|---|---|---|
| Ingredients | Stock (% w/w) | 1.5% stock |
| Lemongrass oil | 3.3 | 0.05 |
| Orange oil | 1.6 | 0.025 |
| Grapefruit seed extract | 15 | 0.225 |
| Multifruit BSC | 13.3 | 0.2 |
| Mixture of lactic, citric, tartaric, | | |
| glycolic, malic acid extracted from | | |
| plants (obtained from Arch | | |
| Chemicals) | | |
| Octanediol | 66.8 | 1.0 |

TABLE 197
Preservative composition -11-G-L

| Ingredients | Stock (% w/w) | Cream containing 1.5% stock |
|---|---|---|
| Lemongrass oil | 5.0 | 0.075 |
| Orange oil | 1.7 | 0.025 |
| Grapefruit seed extract | 13.3 | 0.2 |
| Lactic Acid | 13.3 | 0.2 |
| Octanediol | 66.7 | 1.0 |

TABLE 198
Preservative composition -11-G-M

| | | Cream containing |
|---|---|---|
| Ingredients | Stock (% w/w) | 1.5% stock |
| Lemongrass oil | 5.0 | 0.075 |
| Orange oil | 1.7 | 0.025 |
| Grapefruit seed extract | 13.3 | 0.2 |
| Multifruit BSC | 13.3 | 0.2 |
| Mixture of lactic, citric, tartaric, | | |
| glycolic, malic acid extracted from | | |
| plants (obtained from Arch | | |
| Chemicals) | | |
| Octanediol | 66.7 | 1.0 |

### Example 31

The following example evaluates preservative activity of the formulations noted in preceding examples containing low concentrations of essential oils and botanical extract.

Test Method B. Test inoculums were prepared as follows.
Bacteria: 10⁸ CFU organism/ml.
Yeast (C. albicans): 10⁷ CFU organism/ml.
Fungi (Aspergillus niger): 10⁶ cfu organism/ml.
   For the test samples, preservative was added to 10 grams of the cream at a concentration Of 1.5-2%. and mixed well. From this sample, 1 gram aliquots were placed into 10 ml sterile plastic culture tubes and 0.1 ml (100 micro liters) of the test inoculums was added and vortexed until uniformly blended. The tubes were then placed into incubators under the following temperatures: 30° C. for Aspergillus niger and 37° C. for the remaining three microbes. All tubes were incubated for a total of 1-2 days. At the end of the incubation period, 9.0 ml of Butterfield Phosphate Buffered solution with neutralizer was added to the incubated cultured sample and vortexed until completely mixed. The samples were serially diluted and then plated in Trypticase soy agar (TSA). The plates were incubated at 37° C. temperature for 24-48 hours, and the counts were read. Placebo cream was tested similarly and used as the control. Table 199 shows the results of the testing. In order for effective preservation, the log reduction should be 3 or more within 72 hours.

TABLE 199
Log10 Reduction from control growth

| Preservative | S. aureus | P. aeruginosa | Aspergillus niger |
|---|---|---|---|
| 1 | 7.5 | 7.5 | 4.0 |
| 2 | 7.5 | 7.5 | 4.2 |
| 3 | 7.5 | 7.8 | 4.6 |
| 5 | 7.3 | 7.8 | 3.3 |
| 6 | 7.5 | 7.8 | 2.8 |
| 7 | 7.5 | 7.8 | 2.5 |
| 7A | 7.5 | 7.8 | 2.8 |

The Control had 7.5-7.8 log₁₀ colony counts for bacteria and 4.6 for A. niger. The results of S. aureus and P. aeruginosa are after 24 hours incubation and that of Aspergillus Niger is after 2 days 2% preservative added to the cream.

Conclusion: All the preservatives were effective. However the groups containing phenoxyethanol were more effective in the case of A. niger.

TABLE 200
Log10 Reduction from control (cream with no preservative) growth

| Preservative | S. aureus | P. aeruginosa | Aspergillus niger |
|---|---|---|---|
| G-8 | 7.5 | 7.8 | 2.7 |
| G-10 | 7.3 | 7.8 | 3.4 |
| G_10M | 7.3 | 7.3 | 3.6 |
| G-10C | 7.5 | 7.8 | 3.5 |
| G-11 | 7.5 | 7.8 | 4.5 |
| G-12 | 7.5 | 7.8 | 2.8 |

The results of S. aureus and P. aeruginosa are after 24 hours incubation and that of Aspergillus Niger is after 2 days. 2% preservative added to the cream.

Conclusion: Groups containing Grape fruit seed extract and essential oils are more effective than grapefruit seed extract in the case of A. niger.

TABLE 201
Log10 Reduction from control growth

| Preservative | S. aureus | P. aeruginosa | Aspergillus niger |
|---|---|---|---|
| 6-L | 7.5 | 7.8 | 2..0 |
| 6-M | 7.3 | 7.8 | 2.2 |
| 10-G-L | 6.3 | 7.8 | 2.2 |
| 10-G-M | 7.5 | 7.8 | 2.4 |
| 11-G-L | 7.5 | 7.8 | 2.2 |
| 11-G-M | 7.5 | 7.8 | 2.4 |

The results of S. aureus and P. aeruginosa are after 24 hours incubation and that of Aspergillus Niger is after 2-3 days Figures in Parenthesis are the log reduction after 3 days incubation. 1.5% preservative added to the cream

Conclusion: Multifruit groups are more effective against A. Niger. However, all these groups which do not have solvents are highly effective against bacteria, but slightly less effective against A. Niger. It appears that the solvents releases sufficient amount of oil/extracts to inactivate A. Niger.

### Example 32

The following example evaluates the synergistic activity of essential oils and plant extracts with citric acid against S. aureus.

TABLE 202

| Compounds % | Log₁₀ Reduction |
|---|---|
| 1 Citric acid | 0.7 |
| 0.2 Grape fruit seed extract | 0.64 |
| 0.2 Grape fruit seed extract + 1 Citric acid | 5.91 |
| 0.5 Lemongrass oil + 1 Citric acid | 5.66 |
| 0.3 Lemongrass oil + 0.2 Grape fruit seed | 7.31 |
| extract + 1 Citric acid | |
| 0.25 Manuka oil | 0.94 |
| 0.25 Manuka oil + 1 Citric acid | 5.85 |
| 0.25 Rosemary oil | 0.48 |
| 0.25 Rose Mary oil + 1 Citric acid | 2.0 |
| 0.25 Pomegranate oil | 0.49 |
| 0.25 Pomegranate oil + 1 Citric acid | 7.31 |
| 0.25 Pomegranate extract | 0.40 |
| 0.25 Pomegranate extract + 1 Citric acid | 6.21 |
| 0.25 Calendula oil | 0.50 |
| 0.25 Calendula oil + 1 Citric acid | 3.07 |

Log₁₀ reduction from control bacterial counts that range from 1 × 10⁷ to 5 × 10⁷.

Conclusion: All the essential oils or extracts tested showed synergistic activity with citric acid but Pomegranate oil has more synergistic activity compared to others. Rosemary showed least synergism with citric acid. Lemongrass and grapefruit seed extract together with citric acid had more synergistic activity than lemongrass or grapefruit seed extract alone.

### Example 33

The following example demonstrates the use of glycerine as a solvent in preservative compositions containing low concentrations of essential oil/botanical extract.

The following preservative compositions were prepared and tested for their effectiveness.

TABLE 203
Preservative composition-G-17-G

| Ingredients | Stock (% w/w) | Cream containing 2.5% stock |
|---|---|---|
| Lemongrass oil | 2.8 | 0.07 |
| Grapefruit seed extract | 8.0 | 0.2 |
| Lactic acid | 8.0 | 0.2 |
| Octanediol | 40 | 1.0 |
| Glycerine | 41.2 | 1.03 |

The pH of the stock solution is 5.0. Glycerine is a good alternative solvent for those individuals who have skin sensitivity to propylene glycol, which has been shown to absorb through the skin, or phenoxyethanol, which may be irritating to certain individuals.

The formulation was tested against different organisms for its antimicrobial effects. The data are shown in the following Table.

TABLE 204

| | Log₁₀ reduction from |
|---|---|
| Organism | control bacterial counts |
| A. niger | 4.93 |
| S. aureus | 7.3 |
| P. aureginosa | 7.8 |

The data demonstrate that the preservative formulation containing glycerine as the solvent is also an effective antimicrobial preservative composition.

### Example 34

The following example demonstrates the use of low concentrations of solvents in preservative formulations. The following preservative compositions were prepared.

TABLE 205
Preservative composition - G-18-G

| | Composition of Stock | Cream containing |
|---|---|---|
| Ingredients | Solution (% w/w) | 2.5% stock |
| Lemongrass oil | 4.67 | 0.07 |
| Grapefruit seed extract | 13.33 | 0.2 |
| Lactic acid | 13.33 | 0.2 |
| Octanediol | 46.67 | 0.7 |
| Glycerine | 22.0 | 0.33 |
| (pH of Stock solution 5.0) | | |

TABLE 196
Preservative composition - G-18

| | Composition of Stock | Cream containing |
|---|---|---|
| Ingredients | Solution (% w/w) | 2.5% stock |
| Lemongrass oil | 4.67 | 0.07 |
| Grapefruit seed extract | 13.33 | 0.2 |
| Lactic acid | 13.33 | 0.2 |
| Octanediol | 46.67 | 0.7 |
| Propylene glycol | 22.0 | 0.33 |
| (pH of Stock solution 5.0) | | |

TABLE 197
Preservative composition - G-19-G

| | Composition of Stock | Cream containing |
|---|---|---|
| Ingredients | Solution (% w/w) | 2.5% stock |
| Lemongrass oil | 4.67 | 0.07 |
| Grapefruit seed extract | 13.33 | 0.2 |
| Lactic acid | 13.33 | 0.2 |
| Octanediol | 46.67 | 0.5 |
| Glycerine | 22.0 | 0.53 |
| (pH of Stock solution 5.0) | | |

TABLE 198
Preservative composition - G-19

| | Composition of Stock | Cream containing |
|---|---|---|
| Ingredients | Solution (% w/w) | 2.5% stock |
| Lemongrass oil | 4.67 | 0.07 |
| Grapefruit seed extract | 13.33 | 0.2 |
| Lactic acid | 13.33 | 0.2 |
| Octanediol | 46.67 | 0.5 |
| Propylene glycol | 22.0 | 0.53 |
| (pH of Stock solution 5.0) | | |

### Example 35

The following experiment was carried out to evaluate the synergistic effect of botanical extract, essential oil and fruit acids in a soap base.

Method A: 0.8 gms of plain Softsoap containing the following combination was mixed with 0.1 ml of S. aureus culture (10⁸ cfu/ml) and 0.1 ml bovine serum. After 30 seconds, 9 ml of drug neutralizing media(DNB) was added and mixed. Then, serial dilutions were made with DNB and plated on Trypticase Soy agar.

The stock solution of the soap compositions (LG-6 soaps) is summarized in the following Table. 10-20% of the stock solutions maybe used to prepare specific soap formulations.

TABLE 199
Stock compositions for LG-6 soaps

| Ingredient | % (w/w) |
|---|---|
| Lemongrass oil | 4.29 |
| Grapefruit seed extract | 2.85 |
| Orange oil | 1.42 |
| Alkanediols | 7.14 |
| Fruit Acids | 14.29 |
| Alcohol | 70.0 |

The following soap formulations were prepared.

TABLE 200
LG-6-O

| Ingredient | % (w/w) |
|---|---|
| Lemongrass oil | 0.3 |
| Grapefruit seed extract | 0.2 |
| Orange oil | 0.1 |
| Octanediol | 0.5 |
| SDA 3C | 4.9 |
| Citric Acid | 1.0 |
| Softsoap Base (Colgate Palmolive) | 93.0 |

TABLE 201
LG-6-S

| Ingredient | % (w/w) |
|---|---|
| Lemongrass oil | 0.3 |
| Grapefruit seed extract | 0.2 |
| Orange oil | 0.1 |
| Symclariol | 0.5 |
| Phenoxyethanol | 1.0 |
| SDA 3C | 3.9 |
| Citric Acid | 1.0 |
| Softsoap Base (Colgate Palmolive) | 93.0 |

TABLE 202
LG-6-O-TC

| Ingredient | % (w/w) |
|---|---|
| Lemongrass oil | 0.3 |
| Grapefruit seed extract | 0.2 |
| Orange oil | 0.1 |
| Octanediol | 0.5 |
| SDA 3C | 4.75 |
| Citric Acid | 1.0 |
| Triclosan | 0.15 |
| Softsoap Base (Colgate Palmolive) | 93.0 |

The stock solution of alternative soap compositions (LG-19 Soaps) is summarized in the following Table. 10%-20% of the stock solutions may be used to prepare specific soap formulations.

TABLE 203
Stock compositions for LG-19 soaps

| Ingredient | % (w/w) |
|---|---|
| Lemongrass oil | 1.56 |
| Grapefruit seed extract | 1.04 |
| Orange oil | 0.52 |
| Alkanediols | 2.60 |
| Fruit Acids | 5.21 |
| Alcohol | 89.06 |

The following soap formulations were prepared.

TABLE 204
LG-19-O

| Ingredient | % (w/w) |
|---|---|
| Water | 63.1 |
| Methocil (40-101) | 0.2 |
| U-care Jr | 0.3 |
| Pluronic F-87 | 1.0 |
| Montalene C-40 | 2.0 |
| Incromine oxide L | 8.0 |
| Crosultane C-50 | 3.0 |
| Glycerine | 2.0 |
| Polyoxyl SR-N-60K | 0.2 |
| SDA 40 B | 17.1 |
| Citric acid | 1.0 |
| Lemongrass oil | 0.3 |
| Orange oil | 0.1 |
| Grapefruit seed extract | 0.2 |
| Phenoxy ethanol | 1.0 |
| Octanediol | 0.5 |

TABLE 205
LG-19-S

| Ingredient | % (w/w) |
|---|---|
| Water | 63.1 |
| Methocil (40-101) | 0.2 |
| U-care Jr | 0.3 |
| Pluronic F-87 | 1.0 |
| Montalene C-40 | 2.0 |
| Incromine oxide L | 8.0 |
| Crosultane C-50 | 3.0 |
| Glycerine | 2.0 |
| Polyoxyl SR-N-60K | 0.2 |
| SDA 40 B | 17.1 |
| Citric acid | 1.0 |
| Lemongrass oil | 0.3 |
| Orange oil | 0.1 |
| Grapefruit seed extract | 0.2 |
| Phenoxy ethanol | 1.0 |
| Symclariol | 0.5 |

The following table contains the test data of the LG-6-O composition.

TABLE 206
Effect of LG-6-O against S. aureus

| Compounds | Log₁₀ reduction from control* |
|---|---|
| Dial ® Soap-TC (0.15% TC) | 0.33 |
| LOG 1 | 6.01 |

*Log reduction from control bacterial counts ranged from 1-4 × 10⁷.

Various patent and non-patent publications are cited herein, the contents of which are hereby incorporated by reference in their entireties.

We claim:

1. A preservative composition comprising:
(a) one or more essential oil or individual constituent thereof, wherein each essential oil or individual constituent is present at a concentration between about 0.3 and 15% (weight/weight);
(b) a botanical extract at a concentration between about 0.3 and 30% (weight/weight);
(c) a fruit acid at a concentration between about 5 and 20% (weight/weight); and
(d) an alkanediol at a concentration between about 1 and 80% (weight/weight).

2. The preservative composition of claim 1, wherein the concentration of the essential oil or individual constituent thereof is between about 0.5 and 6.0% (weight/weight).

3. The preservative composition of claim 1, wherein the total concentration of the essential oils and botanical extracts in concentrations is between about 0.5 and about 30% (weight/weight).

4. The preservative composition of claim 3, wherein the total concentration of essential oils and botanical extracts in concentrations is between about 2 and about 20% (weight/weight).

5. The preservative composition of claim 1, wherein the concentration of fruit acids is between about 10 and 20% (weight/weight).

6. The preservative composition of claim 1, wherein the concentration of alkanediols is between about 30 and 80% (weight/weight).

7. The preservative composition of claim 6, wherein the concentration of alkanediols is between about 20 and 50% (weight/weight).

8. The preservative composition of claim 1, further comprising a solvent.

9. The preservative composition of claim 8, wherein the concentration of solvent is between about 0 and 90% (weight/weight).

10. The preservative composition of claim 1, wherein the essential oil and/or constituent thereof is selected from the group consisting of lemongrass oil, an individual constituent of lemongrass oil, orange oil, an individual constituent of orange oil, cinnamon leaf oil, and individual constituent of cinnamon leaf oil, basil oil, and an individual constituent of basil oil, safflower oil, and individual constituent of safflower oil, manuka oil, and an individual constituent of manuka oil, and pomegranate oil, and an individual constituent of pomegranate oil.

11. The preservative composition of claim 1, wherein the fruit acid is selected from the group consisting of lactic acid, citric acid, and multifruit BSC.

12. The preservative composition of claim 1, wherein the botanical extract is selected from the group consisting of grape seed extract, grapefruit seed extract, and pomegranate extract.

13. The preservative composition of claim 1, wherein the alkanediol is octanediol.

14. The preservative composition of claim 8, wherein the solvent is selected from the group consisting of phenoxyethanol, propylene glycol, and glycerine.

15. The preservative composition of claim 1, wherein the composition is suitable for use as a personal care product selected from the group consisting of a bar soap, a liquid hand soap, a hand sanitizer, wound care product, a body wash, an acne treatment, a shampoo, a hair conditioner, a cosmetic, a deodorant, a body lotion, a hand cream, a topical cream, an aftershave lotion, a skin toner, a mouth wash, a toothpaste, a sunscreen lotion, a baby cleansing wipe, a disinfecting wipe, and a diaper cream.

16. The preservative composition of claim 15, wherein the composition is used in concentrations from about 1 to 5% (weight/weight) in personal care products.

17. The preservative composition of claim 1, wherein regular exposure of skin to the composition does not produce skin irritation in a normal subject.

18. An antimicrobial composition comprising:
(a) one or more essential oil or individual constituent thereof, wherein each essential oil or individual constituent is present at a concentration between about 0.5 and 15% (weight/weight);
(b) a botanical extract at a concentration between about 1 and 30% (weight/weight);
(c) a fruit acid at a concentration between about 5 and 20% (weight/weight);
(d) an alkanediol at a concentration between about 1 and 20% (weight/weight); and
(e) a solvent at a concentration between about 0 and 90% (weight/weight).

19. The antimicrobial composition of claim 18, wherein the composition is suitable for use as a personal care product selected from the group consisting of a bar soap, a liquid hand soap, a hand sanitizer, wound care product, a body wash, an acne treatment, a shampoo, a hair conditioner, a cosmetic, a deodorant, a body lotion, a hand cream, a topical cream, an aftershave lotion, a skin toner, a mouth wash, a toothpaste, a sunscreen lotion, a baby cleansing wipe, a disinfecting wipe, and a diaper cream.

20. The antimicrobial composition of claim 19, wherein the composition is used in concentrations from about 10 to about 20% (weight/weight) in personal care products.

21. The antimicrobial composition of claim 18, wherein the solvent is selected from the group consisting of alcohol, phenoxyethanol, propylene glycol, and glycerine.
